(19) United States
(12) Patent Application Publication
(10) Pub. No.: US 2023/0212566 A1
(43) Pub. Date: Jul. 6, 2023

(54) DELIVERY OF GENE EXPRESSION MODULATING AGENTS FOR THERAPY AGAINST CANCER AND VIRAL INFECTION

(71) Applicants: Kiminobu SUGAYA, Orlando, FL (US); Jonhoi SMITH, Orlando, FL (US); UNIVERSITY OF CENTRAL FLORIDA RESEARCH FOUNDATION, INC., Orlando, FL (US)

(72) Inventors: Kiminobu SUGAYA, Orlando, FL (US); Jonhoi SMITH, Orlando, FL (US)

(21) Appl. No.: 17/910,968

(22) PCT Filed: Mar. 10, 2021
(86) PCT No.: PCT/US2021/021674

§ 371 Date: Sep. 12, 2022

Related U.S. Application Data
(60) Provisional application No. 62/987,483, filed on Mar. 10, 2020.

(51) Int. Cl. C12N 15/113; A61P 35/00; A61K 45/06; A61P 31/14; A61K 9/107

(52) U.S. Cl. CPC C12N 15/113; A61K 9/1075; A61K 45/06; A61P 31/14; A61P 35/00; C12N 15/1131; C12N 2310/122; C12N 2310/531; C12N 2320/31; C12N 2320/32

(57) ABSTRACT

Methods and agents that target nanog or Oct4 expression or activity for treating or preventing cancer are disclosed. Alternative methods involve diagnosing cancer stage or type by identifying presence of cancer cells expressing nanog or Oct4. Also, disclosed are method of treating coronavirus infection that involves administering antiviral knockdown agents, such as oligonucleotide-based inhibitors.

### BACKGROUND

Cancer is one of the most significant health conditions. The American Cancer Society’s Cancer Facts and Figures, 2003, predicts over 1.3 million Americans will receive a cancer diagnosis this year. In the United States, cancer is second only to heart disease in mortality accounting for one of four deaths. In 2002, the National Institutes of Health estimated total costs of cancer totaled $171.6 billion, with $61 billion in direct expenditures. The incidence of cancer is widely expected to increase as the US population ages, further augmenting the impact of this condition. The current treatment regimens for cancer, established in the 1970s and 1980s, have not changed dramatically. These treatments, which include chemotherapy, radiation and other modalities including newer targeted therapies, have shown limited overall survival benefit when utilized in most advanced stage common cancers since, among other things, these therapies primarily target tumor bulk rather than cancer stem cells.

More specifically, conventional cancer diagnosis and therapies to date have attempted to selectively detect and eradicate neoplastic cells that are largely fast-growing (i.e., cells that form the tumor bulk). Standard oncology regimens have often been largely designed to administer the highest dose of irradiation or a chemotherapeutic agent without undue toxicity, i.e., often referred to as the “maximum tolerated dose” (MTD) or “no observed adverse effect level” (NOAEL). Many conventional cancer chemotherapies (e.g., alkylating agents such as cyclophosphamide, antimetabolites such as 5-Fluorouracil, plant alkaloids such as vincristine) and conventional irradiation therapies exert their toxic effects on cancer cells largely by interfering with cellular mechanisms involved in cell growth and DNA replication. Chemotherapy protocols also often involve administration of a combination of chemotherapeutic agents in an attempt to increase the efficacy of treatment. Despite the availability of a large variety of chemotherapeutic agents, these therapies have many drawbacks (see, e.g., Stockdale, 1998, “Principles Of Cancer Patient Management” in Scientific American Medicine, vol. 3, Rubenstein and Federman, eds., ch. 12, sect. X). For example, chemotherapeutic agents are notoriously toxic due to non-specific side effects on fast-growing cells whether normal or malignant; e.g. chemotherapeutic agents cause significant, and often dangerous, side effects, including bone marrow depression, immunosuppression, gastrointestinal distress, etc.

Other types of traditional cancer therapies include surgery, hormonal therapy, immunotherapy, epigenetic therapy, anti-angiogenesis therapy, targeted therapy (e.g. therapy directed to a cancer target such as Gleevec®and other tyrosine kinase inhibitors, Velcade®, Sutent®, et al.), and radiation treatment to eradicate neoplastic cells in a patient (see, e.g., Stockdale, 1998, “Principles of Cancer Patient Management,” in Scientific American: Medicine, vol. 3, Rubenstein and Federman, eds., ch. 12, sect. IV). All of these approaches can pose significant drawbacks for the patient including a lack of efficacy (in terms of long-term outcome (e.g. due to failure to target cancer stem cells) and toxicity (e.g. due to non-specific effects on normal tissues)). Accordingly, new therapies and/or regimens for improving the long-term prospect of cancer patients are needed.

Cancer stem cells comprise a unique subpopulation (often 0.1-10% or so) of a tumor that, relative to the remaining 90% or so of the tumor (i.e., the tumor bulk), are more tumorigenic, relatively more slow-growing or quiescent, and often relatively more chemoresistant than the tumor bulk. Given that conventional therapies and regimens have, in large part, been designed to attack rapidly proliferating cells (i.e. those cancer cells that comprise the tumor bulk), cancer stem cells which are often slow-growing may be relatively more resistant than faster growing tumor bulk to conventional therapies and regimens. Cancer stem cells can express other features which make them relatively chemoresistant such as multi-drug resistance and anti-apoptotic pathways. The aforementioned would constitute a key reason for the failure of standard oncology treatment regimens to ensure long-term benefit in most patients with advanced stage cancers--i.e. the failure to adequately target and eradicate cancer stem cells. In some instances, a cancer stem cell(s) is the founder cell of a tumor (i.e., it is the progenitor of the cancer cells that comprise the tumor bulk).

Respiratory Viral infections are primarily initialized as an infection of the respiratory tract. Examples of viruses that infect the respiratory tract are rhinoviruses, influenza viruses (during annual winter epidemics), parainfluenza viruses, respiratory syncytial virus (RSV), enteroviruses, coronaviruses, and certain strains of adenovirus are the main causes of viral respiratory infections.

Coronaviruses and specifically COVID-19 represent new emerging viruses affecting humans and is one type of virus amongst a family of viruses that effect a variety of species. Coronavirus disease 2019 (COVID-19) is defined as illness caused by a novel coronavirus now called severe acute respiratory syndrome coronavirus 2 (SARS-CoV-2; formerly called 2019-nCoV), which was first identified amid an outbreak of respiratory illness cases in Wuhan City, Hubei Province, China. Coronavirus infection in humans is characterized by a broad array of physiologic and anatomical abnormalities that can result in an acute or chronic condition, for example, including altered glucose disposition, hypertension, retinopathy, abnormal kidney function, abnormal central nervous system function, abnormal cardiac function , abnormal liver function, abnormal platelet activity, abnormal pancreatic function aberrations involving large, medium and small sized vessels, chronic fatigue, rhabdomyolysis, and other co-morbidities and death.

### BRIEF DESCRIPTION OF DRAWINGS

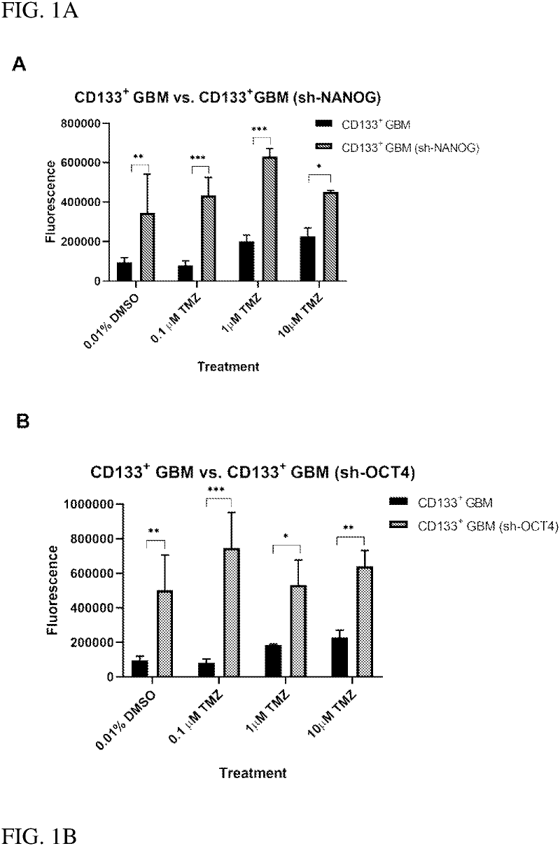

### FIG. 1: TMZ 24-Hours with 0.1, 1, and 10 µM

Cell Viability after 24-hour treatment of cells with TMZ. Cell treatment was performed with TMZ concentration of 0.1, 1, and 10 µM TMZ with 0.01% DMSO solution used as the control. CD133+ GBM non-silenced vs. CD133+ GBM with NANOG expression silenced (A). CD133+ GBM non-silenced vs. CD133+ GBM with OCT4 expression silenced (B). Cell death assessed by measuring fluorescence. Amount of fluorescence is proportional to the amount of cell death that has occurred. 5,000 cells per well was used in this assay. (*p<0.05) (**p<0.01) (*** p<0.001)

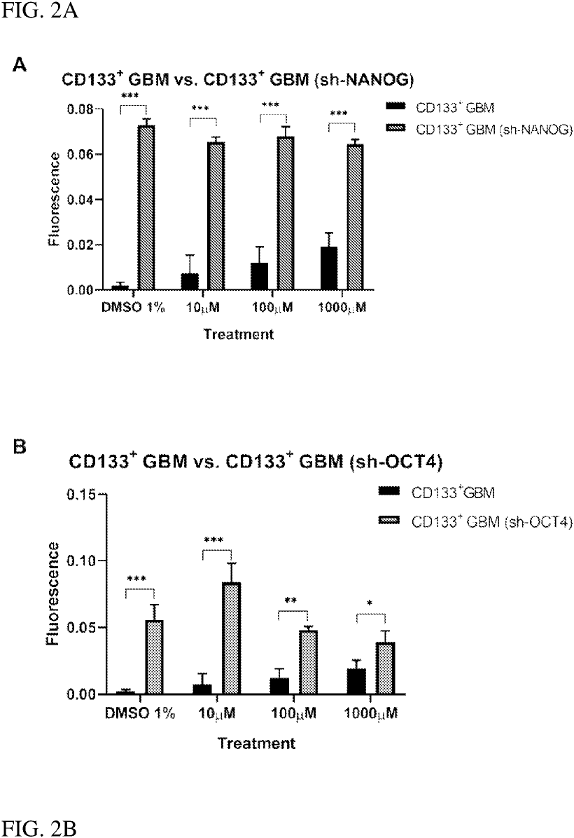

### FIG. 2: TMZ 24-Hours with 10, 100, and 1000 µM

Cell Viability after 24-hour treatment of cells with TMZ. Cell treatment was performed with TMZ concentration of 10, 100, and 1000 µM TMZ with 1% DMSO solution used as the control. CD133+ GBM non-silenced vs. CD133+ GBM with NANOG expression silenced (A). CD133+ GBM non-silenced vs. CD133+ GBM with OCT4 expression silenced (B). Cell death assessed by measuring fluorescence. Amount of fluorescence is proportional to the amount of cell death that has occurred. 100,000 cells per well was used in this assay. (*p<0.05) (**p<0.01) (*** p<0.001).

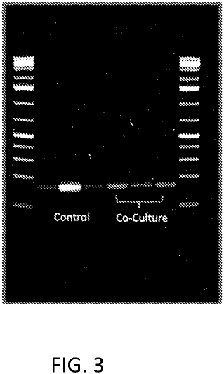

FIG. 3 shows a gel electrophoresis showing detection of HCoV 229E by PCR where MRC-5 (ATCC CCL-171) cells were co-cultured with/without HEK293 cells producing shRNA targeting HCoV 229E genome. The MRC-5 cells were derived from normal lung tissue of a 14 week old male human fetus. Lane 1: Ladder; Lane 2: No-sample; Lane 3: HCoV 229E infected MRC-5 Fibroblast; Lane 4: No-sample; Lane 5-7: HCoV 229E infected MRC-5 Fibroblast co-cultured with HEK293 cells producing shRNA targeting HCoV 229E genome; Lane 8: Ladder

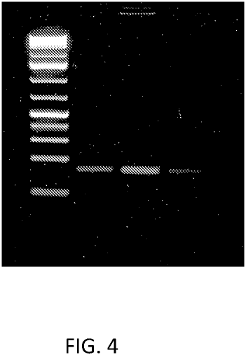

FIG. 4 provides a photograph of a gel electrophoresis showing detection of HCoV 229E by PCR with different kinds of treatment. It shows reduction of HCoV 229E in the cells treated with shRNA targeting HCoV 229E genome. Especially, remarkable reduction was observed in the cells received exosomal delivery of the shRNA. Lane 1: Ladder; Lane 2: HCoV 229E infected MRC-5 Fibroblast (treated with shRNA alone, without exosomes); Lane 3: HCoV 229E infected MRC-5 Fibroblast (treated with exosomes without shRNA); Lane 4: HCoV 229E infected MRC-5 Fibroblast (treated with exosomes with shRNA)

### DETAILED DESCRIPTION

### Overview for Cancer Therapy

In one aspect, the disclosure provides a method of treating cancer in a patient in need thereof, the method comprising administering a therapeutically effective amount of an agent that downregulates the expression or activity of a stemness gene (e.g nanog or Oct4, or both), either directly or indirectly (referred to herein as a “sternness modulating agent”), wherein the patient has been diagnosed with cancer. A non-limiting list of cancers to be treated include urothelial carcinoma, cervical cancer, hematologic cancers, such as leukemia and myeloma, thyroid carcinoma, adenoid cystic carcinoma, breast carcinoma, ovarian cancer, prostate cancer, colon cancer, pancreatic cancer, lymphoma, and neuroblastoma leukemia.

In some embodiments, the patient receives a coadministration of a conventional cancer therapy for the treatment of the cancer before, during or after the administration of the therapeutically effective amount of the stemness modulating agent, such that the effects of the conventional cancer therapy and stemness modulating agent overlap. A non-limiting list of categories of stemness modulating agents for use in compositions and methods described herein includes, shRNA, siRNA or ribozymes that disrupt expression of nanog or Oct4, or transcription factors that modulate expression of nanog or Oct4, or agents that bind directly to nanog or Oct4 that affect their activity. A non-limiting list of examples of such a conventional cancer therapy include chemotherapy, radiation therapy, and/or a combination thereof.

In another aspect, the disclosure provides a method of treating cancer in a patient, the method comprising administering to a patient in need thereof a stemness modulating agent, wherein the patient is in remission for the cancer. In yet other aspects, the patient has been previously treated with conventional chemotherapeutic agents or had radiation therapy. In yet another aspect, the patient can be treated with the stemness modulating agent following, during or prior to the administration of a conventional chemotherapeutic agent or radiation therapy. Further, the cancer can be refractory or multi-drug resistant. In other aspects, the patient can be treated locally with the methods of the disclosure. For example, a bladder cancer patient could be treated with the disclosure via local delivery directly into the tumor, or into the bladder. Local treatment may also be administered in combination, before, or after other local treatments as well (e.g. BCG therapy).

In yet another aspect, provided is a method for preventing a recurrence of cancer in a patient in remission, the method comprising administering to a patient in need thereof a prophylactically effective amount of a stemness modulating agent to the patient. In another aspect, provided is a method for preventing a recurrence of cancer in a patient that has already undergone conventional cancer treatment, the method comprising administering to a patient in need thereof a prophylactically effective amount of a stemness modulating agent.

In another embodiment, the disclosure provides a method for preventing cancer in a patient that is at a high risk for developing cancer, e.g. a patient that has been diagnosed with a nanog-positive and/or Oct4-positive precancerous lesion or may have a genetic or behaviorally influenced propensity for a cancer, the method comprising administering to a patient in need thereof a prophylactically effective amount of a stemness modulating agent.

In a specific aspect, methods can further comprise monitoring the amount of cancer cells or cancer stem cells expressing nanog and/or Oct4 in a patient undergoing cancer treatment. The methods disclosed herein may further comprise determining a course of treatment based on the amount of cancer cells or cancer stem cells expressing nanog and/or Oct4 detected in the patient. The cancer or cancer stem cells may be detected in the patient or in a specimen obtained from the patient. In some embodiments, the specimen is a blood specimen, bone marrow sample, a tissue biopsy, or a tumor biopsy. The amount of cancer cells or cancer stem cells present in the patient or in a sample obtained from the patient can be compared to those present in a reference sample or a sample of cancer cells or cancer stem cells obtained from the patient before or during cancer treatment. In a specific embodiment, the amount of cancer cells or cancer stem cells expressing nanog and/or Oct4 is monitored using an antibody that binds to nanog or Oct4.

In another aspect, provided is a method of treating a solid tumor in a patient, the method comprising administering to a patient in need thereof a therapeutically effective amount of a stemness modulating agent wherein the patient has been diagnosed with a solid tumor, and wherein the patient has undergone convention cancer therapy to reduce the bulk of the tumor.

In particular embodiments of this aspect, the solid tumor is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing’s tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms’ tumor, cervical cancer, uterine cancer, testicular cancer, small cell lung carcinoma, bladder carcinoma, lung cancer, epithelial carcinoma, glioma, glioblastoma multiform, astrocytoma, medulloblastoma, craniopharyngioma, ependymoma, pinealoma, hemangioblastoma, acoustic neuroma, oligodendroglioma, meningioma, skin cancer, melanoma, neuroblastoma, or retinoblastoma.

Also disclosed are antibody conjugates comprising an antibody that binds to nanog or Oct4 linked to a therapeutic agent, a cytotoxic agent or other moiety, and compositions comprising such conjugates and uses of such conjugates, including the treatment of a cancer associated with nanog-expressing and/or Oct4-expressing cells. In some embodiments, an antibody conjugate comprises an agent that is non-proteinaceous, such as a chemotherapeutic agent or radionuclide. In accordance with these embodiments, the agent can be chemically conjugated to the antibody, either directly or through a chemical linker. In other embodiments, an antibody conjugate of the disclosure comprises an agent that is proteinaceous. In accordance with these embodiments, the cytotoxic agent can be covalently linked to the antibody through either a peptide bond or other chemical conjugation. The antibody conjugate can be a recombinantly expressed protein that is generated by the linking via molecular biology techniques of the genes for the antibody (or antibody fragment) with the protein toxin, such that the antibody-conjugate is expressed as a single polypeptide chain containing two domains. Non-limiting examples of agents include diphtheria toxin, Pseudomonas exotoxin, ribosome inactivating proteins, Rnase, ricin A, deglycosylated ricin A chain, abrin, alpha sarcin, aspergillin, restrictocin, ribonucleases, bacterial endotoxin, the lipid A moiety of bacterial endotoxin, bouganin, and cholera toxin. Other examples of cytotoxic agents include, but are not limited to, peptides derived from proteins involved in apoptosis, such as Bcl-x, Bax, or Bad. In one embodiment, the cytotoxic agent is Pseudomonas exotoxin A or a fragment thereof. In a specific embodiment, the cytotoxic agent is a fragment of Pseudomonas exotoxin A that lacks the native receptor binding domain and contains the translocation and ADP-ribosylation domains of Pseudomonas exotoxin A. In another specific embodiment, the cytotoxic agent is a fragment of Pseudomonas exotoxin A that has been modified at its carboxyl terminus so that it has the amino acid sequence Lys-Asp-Glu-Leu (KDEL).

### Overview of Antiviral Therapy

Coronaviruses contain a non-segmented, positive-sense RNA genome of ~30 kb. The genome contains a 5′ cap structure along with a 3′ poly A tail, allowing it to act as an mRNA for translation of the replicase polyproteins. The replicase gene encoding the non-structural proteins (nsps) occupies two-thirds of the genome, about 20 kb, as opposed to the structural and accessory proteins, which make up only about 10 kb of the viral genome. The 5′ end of the genome contains a leader sequence and untranslated region (UTR) that contains multiple stem loop structures required for RNA replication and transcription. Additionally, at the beginning of each structural or accessory gene are transcriptional regulatory sequences (TRSs) that are required for expression of each of these genes. The 3′ UTR also contains RNA structures required for replication and synthesis of viral RNA. The organization of the coronavirus genome is 5′-leader-UTR-replicase-S(Spike)-E(Envelope)-M(Membrane)-N (Nucleocapsid)-3′ UTR-poly (A) tail with accessory genes interspersed within the structural genes at the 3′ end of the genome. This arrangement is exactly same the coronavirus tested herein.

Disclosed herein are studies evidencing that targeting certain viral genes with antiviral knockdown agents and formulating the antiviral knockdown agents for intracellular delivery can destroy coronavirus by targeting their genome. Molecules that bind to coronavirus ribonucleic acid genome sequence include double-stranded or single-stranded DNA, or double-stranded or single-stranded RNA, antisense DNA, CRISPR, phosphorodiamidate morpholino oligomer, antisense RNA, RNA interference (RNAi) molecules (e.g., small interfering RNA (siRNA), and micro-RNA (miRNA), short hairpin RNA (shRNA), etc.).

For example, RNA interference can be used to attack coronavirus genome. RNA interference is the process of sequence-specific post-transcriptional gene silencing in animals mediated by short interfering RNAs (siRNAs). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA. The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Further, described herein is the successful modulation of gene expression implementing delivery of oligonucleotide-based inhibitors by exosomes since they are able to penetrate through tissues and cell membranes. Exosomes are typically 40-150 nm vesicles released by a variety of cell types. Exosomes may be composed of a lipid bilayer and a luminal space containing a variety of proteins, RNAs and other molecules derived from the cytoplasm of the exosome-producing cell. Both the membrane and lumen contents of exosomes may be selectively enriched in subpopulations of lipids, proteins and RNA from the exosome-producing cell. The exosome membrane is frequently, but not necessarily, enriched in lipids including cholesterol and sphingomyelin and contain less phosphatidycholine. The membrane of exosomes may be enriched in particular proteins derived from the plasma membrane of cells.

Combination of these technologies is able to deliver the molecules to destroy or suppress coronavirus RNA genome and stop their propagation or to reduce stemness agents such as nanog and oct4. This invention will very quickly produce effective and innovative therapies for viral infection such as COVID-19 or enable more effective cancer therapy.

### DEFINITIONS

As used herein, the terms “about” or “approximately”, unless otherwise indicated, refer to a value that is no more than 10% above or below the value being modified by the term.

As used herein, the term “sternness modulating agent”, refers to a molecule that decreases expression and/or activity of nanog or Oct4. Specific examples of stemness modulating agents include, but are not limited to, double-stranded or single-stranded DNA, or double-stranded or single-stranded RNA, antisense DNA, phosphorodiamidate morpholino oligomer, antisense RNA, RNA interference (RNAi) molecules (e.g., small interfering RNA (siRNA), and micro-RNA (miRNA), short hairpin RNA (shRNA), etc.), that bind to ribonucleic acid sequence that encodes nanog or Oct4. Stemness modulating agents may also include antibodies or aptamers that bind to nanog or Oct4 and decrease or nullify their activity.

As used herein, the term “antibodies” refer to molecules that contain an antigen binding site, e.g., immunoglobulins. Immunoglobulin molecules can be of any type (e.g., IgG, IgE, IgM, IgD, IgA and IgY), class (e.g., IgG1, IgG2, IgG3, IgG4, IgA1 and IgA2) or subclass. Antibodies include, but are not limited to, monoclonal antibodies, multispecific antibodies, human antibodies, humanized antibodies, camelized antibodies, chimeric antibodies, single domain antibodies, single chain Fvs (scFv), single chain antibodies, Fab fragments, F(ab′) fragments, disulfide-linked Fvs (sdFv), and anti-idiotopic (anti-Id) antibodies (including, e.g., anti-Id antibodies to antibodies of the invention), and epitope-binding fragments of any of the above. The term antibody will include any protein sequence that confers specificity or binding to its target epitope. Any use of the term antibody will include these permutations. Specific examples of antibodies known to bind to nanog include those available from Santa Cruz biotechnology, Inc. (catalogue nos. sc-33759, sc-81961, sc-30329, sc-33760, sc-30331, sc-30332, or sc-30328)

As used herein, the terms “antibody conjugate(s)” and “antibody fragment conjugate(s)” refer to a conjugate(s) of an antibody or antibody fragment that is prepared by way of a synthetic chemical reaction(s) or as a recombinant fusion protein(s). The term antibody conjugate includes any domain or sequence from an antibody that confers specificity for binding its target, including, but not limited to the permutations described in the definition for “antibody” above.

As used herein, the term “bind” or “bind(s)” refers to any interaction, whether direct or indirect, that affects the specified receptor or receptor subunit.

As used herein, the term “cancer” refers to a neoplasm or tumor resulting from abnormal uncontrolled growth of cells. The term “cancer” encompasses a disease involving both pre-malignant and malignant cancer cells. In some embodiments, cancer refers to a localized overgrowth of cells that has not spread to other parts of a subject, i.e., a benign tumor. In other embodiments, cancer refers to a malignant tumor, which has invaded and destroyed neighboring body structures and spread to distant sites. In yet other embodiments, the cancer is associated with a specific cancer antigen.

As used herein, the term “cancer cells” refers to cells that acquire a characteristic set of functional capabilities during their development, including the ability to evade apoptosis, self-sufficiency in growth signals, insensitivity to anti-growth signals, tissue invasion/metastasis, significant growth potential, and/or sustained angiogenesis. The term “cancer cell” is meant to encompass both pre-malignant and malignant cancer cells.

As used herein, the term “cancer stem cell(s)” refers to a cell that can be a progenitor of a highly proliferative cancer cell. A cancer stem cell has the ability to re-grow a tumor as demonstrated by its ability to form tumors in immunocompromised mice, and typically to form tumors upon subsequent serial transplantation in immunocompromised mice. Cancer stem cells are also typically slow-growing relative to the bulk of a tumor; that is, cancer stem cells are generally quiescent. In certain embodiments, but not all, the cancer stem cell may represent approximately 0.1 to 10% of a tumor.

As used herein, the term “chemotherapeutic agent” refers to any molecule, compound, and/or substance that is used for the purpose of treating and/or managing cancer. Chemotherapeutic agents may be agents that achieve anti-angiogenesis therapy, targeted therapy, radioimmunotherapy, small molecule therapy, biologic therapy, epigenetic therapy, toxin therapy, differentiation therapy, pro-drug activating enzyme therapy, antibody therapy, chemotherapy, radiation therapy, hormonal therapy, immunotherapy, or protein therapy. Examples of chemotherapeutic agents include antimetabolites (e.g., cytosine arabinoside, aminopterin, methotrexate, 6-mercaptopurine, 6-thioguanine, cytarabine, and 5-fluorouracil decarbazine); alkylating agents (e.g., mechlorethamine, thiotepa chlorambucil, melphalan, carmustine (BCNU) and lomustine (CCNU), cyclophosphamide, busulfan, dibromomannitol, streptozotocin, mitomycin C, cis-dichlorodiammine-platinum (II) (CDDP), and cisplatin); vinca alkaloid; anthracyclines (e.g., daunorubicin (formerly daunomycin) and doxorubicin); antibiotics (e.g., dactinomycin (formerly actinomycin), bleomycin, mithramycin, and anthramycin (AMC)); calicheamicin; CC-1065 and derivatives thereof; auristatin molecules (e.g., auristatin PHE, bryostatin-1, and dolastatin-10; see Woyke, et al., Antimicrob Agents Chemother 46:3802-8 (2002), Woyke, et al., Antimicrob Agents Chemother 45:3580-4 (2001), Mohammad, et al., Anticancer Drugs 12:735-40 (2001), Wall, et al., Biochem Biophys Res Commun 266:76-80 (1999), Mohammad, et al., Int J Oncol 15:367-72 (1999), all of which are incorporated herein by reference); DNA-repair enzyme inhibitors (e.g., etoposide or topotecan); kinase inhibitors (e.g., compound ST1571, imatinib mesylate (Kantarjian, et al., Clin Cancer Res 8(7):2167-76 (2002)); demecolcine; and other cytotoxic agents (e.g., paclitaxel, cytochalasin B, gramicidin D, ethidium bromide, emetine, mitomycin, etoposide, tenoposide, vincristine, vinblastine, colchicine, doxorubicin, daunorubicin, dihydroxy anthracenedione, mitoxantrone, mithramycin, actinomycin D, 1-dehydrotestosterone, glucorticoids, procaine, tetracaine, lidocaine, propranolol, and puromycin and analogs or homologues thereof and those compounds disclosed in U.S. Pat. Nos. 6,245,759, 6,399,633, 6,383,790, 6,335,156, 6,271,242, 6,242,196, 6,218,410, 6,218,372, 6,057,300, 6,034,053, 5,985,877, 5,958,769, 5,925,376, 5,922,844, 5,911,995, 5,872,223, 5,863,904, 5,840,745, 5,728,868, 5,648,239, 5,587,459); farnesyl transferase inhibitors (e.g., R115777, BMS-214662, and those disclosed by, for example, U.S. Pat. Nos. 6,458,935, 6,451,812, 6,440,974, 6,436,960, 6,432,959, 6,420,387, 6,414,145, 6,410,541, 6,410,539, 6,403,581, 6,399,615, 6,387,905, 6,372,747, 6,369,034, 6,362,188, 6,342,765, 6,342,487, 6,300,501, 6,268,363, 6,265,422, 6,248,756, 6,239,140, 6,232,338, 6,228,865, 6,228,856, 6,225,322, 6,218,406, 6,211,193, 6,187,786, 6,169,096, 6,159,984, 6,143,766, 6,133,303, 6,127,366, 6,124,465, 6,124,295, 6,103,723, 6,093,737, 6,090,948, 6,080,870, 6,077,853, 6,071,935, 6,066,738, 6,063,930, 6,054,466, 6,051,582, 6,051,574, and 6,040,305); topoisomerase inhibitors (e.g., camptothecin, irinotecan, SN-38, topotecan, 9-aminocamptothecin, GG211 (GI147211), DX-8951f, IST-622, rubitecan, pyrazoloacridine, XR5000, saintopin, UCE6, UCE1022, TAN-1518A, TAN 1518B, KT6006, KT6528, ED-110, NB-506, ED-110, NB-506, and rebeccamycin); bulgarein; DNA minor groove binders such as Hoechst dye 33342 and Hoechst dye 33258; nitidine; fagaronine; epiberberine; coralyne; beta-lapachone; BC-4-1; antisense oligonucleotides (e.g., those disclosed in the U.S. Pat. Nos. 6,277,832, 5,998,596, 5,885,834, 5,734,033, and 5,618,709); adenosine deaminase inhibitors (e.g., fludarabine phosphate and 2-chlorodeoxyadenosine); and pharmaceutically acceptable salts, solvates, clathrates, and prodrugs thereof.

The term “co-administration” or “co-administering” as used herein refer to the administration of a substance before, concurrently, or after the administration of another substance such that the biological effects of either substance overlap.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”. “having”, “has”, “with”, or variants thereof are used in either the detailed description and/or the claims, such terms are intended to be inclusive in a manner similar to the term “comprising.”

By “oligonucleotide-based inhibitor” is meant an RNA or DNA molecule that binds to a target nucleic acid that inhibits or disrupts expression of the gene product, or activity of the gene product encoded by the target nucleic acid. Such molecules include, for example, antisense RNA and/or DNA molecules, interference RNA (RNAi), microRNA, or ribozymes. As such, these compounds may be introduced in the form of single-stranded, double-stranded, partially single-stranded, or circular oligomeric compounds.

In the context of this invention, the term “oligonucleotide” refers to an oligomer or polymer of ribonucleic acid (RNA) or deoxyribonucleic acid (DNA) or mimetics thereof. The term “oligonucleotide”, also includes linear or circular oligomers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like. Oligonucleotides are capable of specifically binding to a target polynucleotide by way of a regular pattern of monomer-to-monomer interactions, such as Watson-Crick type of base pairing, Hoogsteen or reverse Hoogsteen types of base pairing, or the like.

The oligonucleotide may be “chimeric”, that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide in the case of an oligonucleotides compound. These oligonucleotides typically comprise at least one region wherein the oligonucleotide is modified in order to exhibit one or more desired properties. The desired properties of the oligonucleotide include, but are not limited, for example, to increased resistance to nuclease degradation, increased cellular uptake, and/or increased binding affinity for the target nucleic acid. Different regions of the oligonucleotide may therefore have different properties. The chimeric oligonucleotides of the present invention can be formed as mixed structures of two or more oligonucleotides, modified oligonucleotides, oligonucleosides and/or oligonucleotide analogs as described above.

As used herein, the term “target nucleic acid” encompasses DNA, RNA (comprising premRNA and mRNA) transcribed from such DNA, and also cDNA derived from such RNA, coding, noncoding sequences, sense or antisense polynucleotides. The specific hybridization of an oligomeric compound with its target nucleic acid interferes with the normal function of the nucleic acid. This modulation of function of a target nucleic acid by compounds, which specifically hybridize to it, is generally referred to as “antisense”.

Selection of appropriate oligonucleotides is facilitated by using computer programs that automatically align nucleic acid sequences and indicate regions of identity or homology. Such programs are used to compare nucleic acid sequences obtained, for example, by searching databases such as GenBank or by sequencing PCR products. Comparison of nucleic acid sequences from a range of species allows the selection of nucleic acid sequences that display an appropriate degree of identity between species. In the case of genes that have not been sequenced, Southern blots are performed to allow a determination of the degree of identity between genes in target species and other species. By performing Southern blots at varying degrees of stringency, as is well known in the art, it is possible to obtain an approximate measure of identity. These procedures allow the selection of oligonucleotides that exhibit a high degree of complementarity to target nucleic acid sequences in a subject to be controlled and a lower degree of complementarity to corresponding nucleic acid sequences in other species. One skilled in the art will realize that there is considerable latitude in selecting appropriate regions of genes for use in the present disclosure.

By “enzymatic RNA” or “ribozyme” is meant an RNA molecule with enzymatic activity. Enzymatic nucleic acids (ribozymes) act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through base pairing, and once bound to the correct site, acts enzymatically to cut the target RNA.

As used herein, “hybridization” means the pairing of substantially complementary strands of oligomeric compounds. One mechanism of pairing involves hydrogen bonding, which may be Watson-Crick, Hoő̈gsteen or reversed Hoögsteen hydrogen bonding, between complementary nucleoside or nucleotide bases (nucleotides) of the strands of oligomeric compounds. For example, adenine and thymine are complementary nucleotides which pair through the formation of hydrogen bonds. Hybridization can occur under varying circumstances.

As used herein, the term “compound” refers to any agent that is being tested for its ability to bind to nanog or Oct4 or has been identified as binding to nanog or Oct4, including the particular antibodies provided herein or incorporated by reference herein. In one embodiment, a compound is purified (e.g., 85%, 90%, 95%, 99%, or 99.9% pure). Such compounds for example, generally include any agent comprised of two or more atoms or ions of two or more elements in chemical combination wherein the constituents are united by bonds or valence forces (see Hawley’s Condensed Chemical Dictionary, Thirteenth Edition, 1997). Non-limiting examples of compounds include, but are not limited to, proteinaceous molecules, including, but not limited to, peptides (including dimers and multimers of such peptides), polypeptides, proteins, including post-translationally modified proteins, conjugates, antibodies, antibody fragments, antibody conjugates, small molecules, including inorganic or organic compounds; nucleic acid molecules including, but not limited to, double-stranded or single-stranded DNA, or double-stranded or single-stranded RNA, antisense RNA, RNA interference (RNAi) molecules (e.g., small interfering RNA (siRNA), micro-RNA (miRNA), short hairpin RNA (shRNA), etc.), intron sequences, triple helix nucleic acid molecules and aptamers; carbohydrates; and lipids.

As used herein, the term “cytotoxin” or the phrase “cytotoxic agent” refers to an antibody that exhibits an adverse effect on cell growth or viability. Included in this definition are compounds that kill cells or which impair them with respect to growth, longevity, or proliferative activity.

As used herein, the phrase “diagnostic agent” refers to any molecule, compound, and/or substance that is used for the purpose of diagnosing cancer. Non-limiting examples of diagnostic agents include antibodies, antibody fragments, or other proteins, including those conjugated to a detectable agent. As used herein, the term “detectable agents” refer to any molecule, compound and/or substance that is detectable by any methodology available to one of skill in the art. Non-limiting examples of detectable agents include dyes, gases, metals, or radioisotopes.

As used herein, the terms “reduce” and “inhibit” are used together because it is recognized that, in some cases, a decrease can be reduced below the level of detection of a particular assay. As such, it may not always be clear whether the expression level or activity is “reduced” below a level of detection of an assay, or is completely “inhibited.”

As used herein, “treatment” or “treating” means to administer a composition to a subject or a system with an undesired condition. The condition can include a disease (including infection) or disorder. “Prevention” or “preventing” means to administer a composition to a subject or a system at risk for the condition, and therefore includes preventing disease progression in symptomatic or asymptomatic subjects. The condition can include a predisposition to a disease or disorder. The effect of the administration of the composition to the subject (either treating and/or preventing) can be, but is not limited to, the cessation of one or more symptoms of the condition, a reduction or prevention of one or more symptoms of the condition, a reduction in the severity of the condition, the complete ablation of the condition, a stabilization or delay of the development or progression of a particular event or characteristic, or minimization of the chances that a particular event or characteristic will occur.

As used herein, the term “therapeutically effective amount” in the context of cancer refers to the amount of a therapy that is sufficient to result in the prevention of the development, recurrence, or onset of cancer and one or more symptoms thereof, to enhance or improve the prophylactic effect(s) of another therapy, reduce the severity, the duration of cancer, ameliorate one or more symptoms of cancer, prevent the advancement of cancer, cause regression of cancer, and/or enhance or improve the therapeutic effect(s) of another therapy. Typically, an effective amount is provided according to a regimen. In an embodiment, the amount of a therapy is effective to achieve one, two, three or more of the following results following the administration of one, two, three or more therapies: (1) a stabilization, reduction or elimination of the cancer stem cell population; (2) a stabilization, reduction or elimination in the cancer cell population; (3) a stabilization or reduction in the growth of a tumor or neoplasm; (4) an impairment in the formation of a tumor; (5) eradication, removal, or control of primary, regional and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) a decrease in hospitalization rate; (10) a decrease in hospitalization lengths; (11) the size of the tumor is maintained and does not increase or increases by less than 10%, preferably less than 5%, preferably less than 4%, preferably less than 2%; (12) an increase in the number of patients in remission; (13) an increase in the length or duration of remission; (14) a decrease in the recurrence rate of cancer; (15) an increase in the time to recurrence of cancer; and (16) an amelioration of cancer-related symptoms and/or quality of life. The term prophylactically effective amount refers to an effective amount administered to a subject either at risk of having cancer or who has already been treated for cancer and is administered to reduce relapse.

As used herein in the context of viral infection, the term “therapeutically effective amount” refers to an amount of a composition of the disclosure that when administered to a human subject in need thereof, is sufficient to effect treatment or prophylaxis for virus infection. The amount that is therapeutically effective will depend upon the patient’s size and gender, the stage and severity of the infection and the result sought. The full therapeutic effect does not necessarily occur by administration of one dose and may occur only after administration of a series of doses. Thus, a therapeutically effective amount may be administered in one or more administrations per day for successive days. For a given patient and condition, a therapeutically effective amount can be determined by methods known to those of skill in the art. For example, in reference to the treatment of a Sars-CoV2 virus infection using the compositions of the present disclosure, a therapeutically effective amount refers to that amount of the composition which has the effect of (1) reducing the shedding of the virus, (2) reducing the duration of the infection, (3) reducing infectivity and/or, (4) reducing the severity (or, preferably, eliminating) one or more other symptoms associated with the infection such as, for example, fever, headache, fatigue, dry cough, sore throat, respiratory distress, muscle aches, conjunctivitis, runny and/or stuffy nose. Such an effective dose will generally depend on the factors described above. A prophylactically effective dose is one that reduces the likelihood of contracting a virus infection.

As used herein, the terms “subject” and “patient” are used interchangeably. As used herein, the term “subject” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is an elderly human. In another embodiment, the subject is a human adult. In another embodiment, the subject is a human child. In yet another embodiment, the subject is a human infant.

In some embodiments of the above-described aspects, the methods involving administering a sternness modulating agent or a chemotherapy or an antiviral knockdown agent can be provided according to a regimen. The term effective amount includes administration according to a regimen. Thus, the term regimen as used herein is encompassed by the term effective amount, but provides more specifics concerning the dosing, frequency and duration of the effective amount, whether the regimen be tailored for therapeutic purposes (therapeutically effective regimen for treating cancer) or prophylactic purposes (prophylactically effective regimen). For example, a regimen can comprise the administration of a stemness modulating agent over a period of 1 to 6 weeks, 1 to 3 months, 3 to 6 months, 1 to 12 months, or 6 to 12 months. In some other embodiments the regimens comprise the administration of a stemness modulating agent over a longer period of time such as 9, 12, 24, 36, or 48 months or for the remainder of the patient’s life.

As used herein, the terms “cancer therapies” and “cancer therapy” can refer to any method(s), useful for the treatment of a cancer or one or more symptoms thereof. In certain embodiments, the terms “therapy” and “therapies” refer to chemotherapy and/or radiation therapy, radioimmunotherapy, hormonal therapy, targeted therapy, toxin therapy, pro-drug activating enayme therapy, protein therapy, antibody therapy, small molecule therapy, epigenetic therapy, demethylation therapy, histone deacetylase inhibitor therapy, differentiation therapy, antiangiogenic therapy, biological therapy including immunotherapy and/or other therapies useful in the treatment of a cancer or one or more symptoms thereof. In a specific embodiment, a therapy is administration of an effective

The terms “treat” “treating” or “treatment of” as used herein refers to providing any type of medical management to a subject. Treating includes, but is not limited to, administering a composition comprising one or more active agents to a subject using any known method. for purposes such as curing, reversing, alleviating, reducing the severity of, inhibiting the progression of, or reducing the likelihood of a disease, disorder, or condition or one or more symptoms or manifestations of a disease, disorder or condition. The administration of the drug can be oral, nasal, parental, topical, ophthalmic, or transdermal administration or delivery in the form of solid, semi-solid, lyophilized powder, or liquid dosage forms. The dosage forms include tablets, capsules, troches, powders, solutions, suspensions, suppositories, or the like, preferably in unit dosage forms suitable for simple administration of precise dosages.

In the context of cancer, the terms “treat”, “treatment”, and “treating” can more specifically refer to the reduction or inhibition of the progression and/or duration of cancer, reduction of risk of developing cancer, the reduction or amelioration of the severity of cancer, and/or the amelioration of one or more symptoms thereof resulting from the administration of one or more therapies. In a specific embodiment, a patient that is at a high risk for developing cancer is treated, i.e., a patient that has been diagnosed with a nanog positive precancerous lesion. In specific embodiments, such terms refer to one, two, or three or more results following the administration of one, two, three or more therapies: (1) a stabilization, reduction or elimination of the cancer stem cell population; (2) a stabilization, reduction or elimination in the cancer cell population; (3) a stabilization or reduction in the growth of a tumor or neoplasm; (4) an impairment in the formation of a tumor; (5) eradication, removal, or control of primary, regional and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) a decrease in hospitalization rate; (10) a decrease in hospitalization lengths; (11) the size of the tumor is maintained and does not increase or increases by less than 10%, preferably less than 5%, preferably less than 4%, preferably less than 2%; (12) an increase in the number of patients in remission; (13) an increase in the length or duration of remission; (14) a decrease in the recurrence rate of cancer; (15) an increase in the time to recurrence of cancer; and (16) an amelioration of cancer-related symptoms and/or quality of life. In certain embodiments, such terms refer to a stabilization or reduction in the cancer stem cell population. In some embodiments, such terms refer to a stabilization or reduction in the growth of cancer cells. In some embodiments, such terms refer to a stabilization or reduction in the cancer stem cell population and a reduction in the cancer cell population. In some embodiments, such terms refer to a stabilization or reduction in the growth and/or formation of a tumor. In some embodiments, such terms refer to the eradication, removal, or control of primary, regional, or metastatic cancer (e.g., the minimization or delay of the spread of cancer). In some embodiments, such terms refer to a reduction in mortality and/or an increase in survival rate of a patient population. In further embodiments, such terms refer to an increase in the response rate, the durability of response, or number of patients who respond or are in remission. In some embodiments, such terms refer to a decrease in hospitalization rate of a patient population and/or a decrease in hospitalization length for a patient population.

Compositions are described that include a stemness modulating agent and/or chemotherapeutic agent. Composition embodiments can be in the form of a solid, liquid or gas (aerosol). Typical routes of administration may include, without limitation, oral, topical, parenteral, sublingual, rectal, vaginal, ocular, intradermal, intratumoral, intracerebral, intrathecal, and intranasal. Parenteral administration includes subcutaneous injections, intravenous, intramuscular, intraperitoneal, intrapleural, intrasternal injection, directly into the lumen of the bladder, directly into the tumor, or infusion techniques. In a specific embodiment, the compositions are administered parenterally. In a more specific embodiment, the compositions are administered intravenously. Pharmaceutical compositions of the disclosure can be formulated so as to allow an antibody of the disclosure to be bioavailable upon administration of the composition to a subject. Compositions can take the form of one or more dosage units, where, for example, a tablet can be a single dosage unit, and a container of an antibody of the disclosure in aerosol form can hold a plurality of dosage units.

Cancer TherapiesFurthermore, different cells in a tumor sample may be isolated based on their histological or growth characteristics. For example, cells from a tumor sample may be adherent to surfaces compared to other cells. Adherent cells are in most cases more differentiated tumor cells not cancer stem cells. Cancer stem cells also may have a propensity to form spheres. Cells tending to form spheres can be selected apart from cells not tending to form spheres. Cells may also be isolated based on the hanging-drop method. Tissue Engineering, Second Edition, Hauser and Fussenegger, 2007, Human Press.

In another embodiment, methods are disclosed for stabilizing, reducing or eliminating a cancer stem cell population. In particular, the present disclosure provides methods for stabilizing, reducing or eliminating a cancer stem cell population in a subject, the method comprising administering to a subject in need thereof a prophylactically or therapeutically effective amount of a stemness modulating agent and optionally co-administering a chemotherapeutic agent. The administering of the stemness modulating agent and/or chemotherapeutic agent is typically conducted according to a regimen. In certain embodiments, the administration of the stemness modulating agent results in the stabilization of a cancer stem cell population as assessed by methods after a period and/or duration of certain survival endpoints. Thus, in order to achieve stabilization, reduction, or elimination in the growth, size, and/or formation of a tumor and/or metastases by stabilizing, reducing or eliminating the cancer stem cell population, a stemness modulating agent and chemotherapeutic agent can be administered for a longer period of time, and in some embodiments, more frequently or more continuously than currently administered or known to one of skill in the art. In certain embodiments, a lower dose than currently used or known to one of skill in the art is administered for a longer period of time, and in some embodiments, more frequently or more continuously than currently administered or known to one of skill in the art.

In other embodiments, the present disclosure provides methods for stabilizing, reducing, or eliminating the cancer stem cells and the cancer cells in a subject, the method comprising administering to a subject in need thereof a prophylactically or therapeutically effective regimen, the regimen comprising administering one or more therapies to the subject. In one embodiment, the regimen achieves a 5%-40%, preferably a 10%-60%, and more preferably a 20 to 99% reduction in the cancer stem cell population, and/or a 5%-40%, preferably a 10%-60%, and more preferably at 20 to 99% reduction in the cancer cell population. In a specific embodiment, the reduction in the cancer stem cell population and/or the cancer cell population is achieved after two weeks, a month, two months, three months, four months, six months, nine months, 1 year, 2 years, 3 years, 4 years, or more of administration of one or more therapies.

The present disclosure provides methods for stabilizing or reducing the population of cancer stem cells and the bulk size of a tumor in a subject, the methods comprising administering to a subject in need thereof a prophylactically or therapeutically effective regimen, the regimen comprising administering one or more therapies to the subject. Typically, the one or more therapies comprises administering an effective amount of at least one stemness modulating agent. In one embodiment, the regimen achieves a 5%-40%, preferably a 10%-60%, and more preferably a 20 to 99% reduction in the cancer stem cell population, and/or a 5%-40%, preferably a 10%-60%, and more preferably a 20 to 99% reduction in the bulk size of the tumor. In a specific embodiment, the reduction in the cancer stem cell population and/or tumor size is achieved after two weeks, a month, two months, three months, four months, six month, nine months, 1 year, 2 years, 3 years, 4 years, or more of administration of one or more of the therapies. In a of time (e.g., after 2, 5, 10, 20, 30 or more doses of a therapy, or after 2 weeks, 1 month, 2 months, 1 year, 2 years, 3 years, 4 years or more). In other embodiments, the regimen achieves a 5%-40%, preferably a 10%-60%, and more preferably a 20 to 99% reduction in the cancer stem cell population. In some embodiments, the reduction in a cancer stem cell population is achieved after two weeks, a month, two months, three months, four months, six month, nine months, 1 year, 2 years, 3 years, or 4 years of administration of one or more therapies. In certain embodiments, in accordance with the regimen, the reduction in a cancer stem cell population is monitored periodically (e.g., after 2, 5, 10, 20, 30 or more doses of one or more therapies, or after 2 weeks, 1 month, 2 months, 1 year, 2 years, 3 years, 4 years or more after receiving one or more therapies).

Without being bound by a particular theory or mechanism, the stabilization, reduction or elimination of a cancer stem cell population stabilizes, reduces or eliminates the cancer cell population produced by the cancer stem cell population, and thus, stabilizes, reduces or eliminates the growth of a tumor, the bulk size of a tumor, the formation of a tumor and/or the formation of metastases. In other words, the stabilization, reduction or elimination of the cancer stem cell population prevents the formation, reformation or growth of a tumor and/or metastases by cancer cells.

Cancer stem cells can proliferate relatively slowly so that conventional therapies and regimens that differentially impair, inhibit or kill rapidly proliferating cell populations (e.g., cancer cells comprising the tumor bulk) in comparison with cell populations that divide more slowly, most likely do not effectively target and impair cancer stem cells. The methods and regimens of the present disclosure are designed to result in a concentration (e.g., in blood, plasma, serum, tissue, and/or tumor) of a therapy(ies) that will stabilize or reduce a cancer stem cell population.

Since cancer stem cells often make up only a subpopulation of a tumor, a therapy that stabilizes, reduces or eliminates cancer stem cells may require a longer period of time than is traditionally expected for a cancer patient to achieve stabilization, reduction or elimination in the growth, size and/or formation of a tumor and/or metastases, or an amelioration of cancer-related symptoms. Accordingly, during this additional time period, there is an opportunity to deliver additional therapy, albeit at less toxic (e.g., lower) doses. As a result of stabilizing, reducing, or eliminating the cancer stem cell population, the cancer may be significantly impaired, the frequency of responses increased albeit potentially occurring at later time points, the duration of a remission increased, and/or the frequency particular embodiment, the reduction in the cancer stem cell population is determined by a method described, infra, and the bulk size of the tumor is measured by methods known to one of skill in the art. Non-limiting examples of methods for measuring the bulk size of a tumor include radiological methods (e.g., computed tomography (CT), MRI, X-ray, mammogram, PET scan, radionuclide scan, bone scan), visual methods (e.g., colonoscopy, bronchoscopy, endoscopy), physical exam (e.g., prostate, breast, lymph nodes, abdominal, general palpation), blood tests (e.g., PSA, CEA, CA-125, AFP, liver function tests), bone marrow analysis (e.g., in the case of a hematological malignancy), histopathology, cytology, and flow cytometry. In certain embodiments, in accordance with the regimen, the cancer stem cell population and/or the tumor size are monitored periodically (e.g., after 2, 5, 10, 20, 30, or more doses of one or more of the therapies, or after 2 weeks, 1 month, 2 months, 6 months, 1 year, or more of receiving one or more therapies).

In certain embodiments, the prophylactically and/or therapeutically effective regimens do not affect tumor angiogenesis. In other embodiments, the prophylactically and/or therapeutically effective regimens reduce tumor angiogenesis by less than 25%, preferably less than 15%, and more preferably less than 10%. Tumor angiogenesis can be assessed by techniques known to one of skill in the art, including, e.g., assessing microvessel density of a tumor and measuring the cancer stem cell population and the cancer stem cell population in a blood sample.

The present disclosure provides methods for stabilizing, reducing, or eliminating the population of cancer stem cells in a subject, the methods comprising administering to a subject in need one or more therapies comprising administering an effective amount of at least one stemness modulating agent. In one embodiment, the regimen achieves 5%-40%, preferably a 10%-60%, and more preferably a 20 to 99% reduction in the cancer stem cell population and less than a 25%, preferably less than a 15%, and more preferably less than a 10% reduction in the cancer stem cell population. In a specific embodiment, the reduction in the cancer stem cell population is achieved after two weeks, a month, two months, three months, four months, six month, nine months, 1 year, 2 years, 3 years, 4 years or more of administration of one or more of the therapies. The present disclosure provides methods for stabilizing, reducing, or eliminating the population of cancer stem cells in a subject, the methods comprising administering to a subject in need thereof a prophylactically or therapeutically effective regimen, the regimen comprising administering one or more therapies to the subject, wherein the regimen does not result in a reduction or results in a small reduction in the cancer stem cell population.

The present disclosure provides methods for preventing, treating and/or managing cancer, the methods comprising administering to a subject in need thereof a prophylactically or therapeutically effective regimen, the regimen comprising administering one or more therapies to the subject, wherein the regimen results in at least an approximately 2.5%, 5%, 10%, 15%, 20%, 25%, 30%, 40%, 50%, 60%, 70%, 80%, 90%, 95%, 97.5%, or 99% reduction in the cancer stem cell population, and the one or more therapies includes administering an effective amount of at least one stemness modulating agent. In one embodiment, the regimen achieves a 5%-40%, preferably a 10%-60%, and more preferably a 20 to 99% reduction in the cancer stem cell population. In a specific embodiment, the reduction in the cancer stem cell population is determined by a method described herein. In some embodiments, the reduction in the cancer stem cell population is achieved after two weeks, a month, two months, three months, four months, six month, nine months, 1 year, 2 years, 3 years, 4 years or more of administration of one or more of the therapies. In certain embodiments, in accordance with the regimen, the reduction in the cancer stem cell population is monitored after a period of time (e.g., after 2, 5, 10 or more doses of one or more of the therapies or after 2 weeks, 1 month, 2 months, 6 months, 1 year, or more of receiving one or more therapies).

The present disclosure provides methods of preventing, treating and/or managing cancer, the method comprising: (a) administering to a subject in need thereof one or more doses of an effective amount of a therapy; (b) monitoring the cancer stem cell population in the subject prior to, during, and/or after the administration of a certain number of doses and prior to the administration of a subsequent dose; and (c) maintaining at least a 5%-40%, preferably a 10%-60%, and more preferably a 20 to 99% reduction in the cancer stem cell population in the subject by repeating step (a) as necessary. In a specific embodiment, the reduction in the cancer stem cell population is determined by a method described, infra. In some embodiments, the reduction of the cancer stem cell population is achieved after 5 to 30, 10 to 50, 10 to 75, 10 to 100, 10 to 150, or 10 to 300 doses of the therapy.

The present disclosure provides methods for preventing, treating and/or managing cancer, the methods comprising administering to a subject in need thereof a prophylactically or therapeutically effective regimen, the regimen comprising administering one or more therapies to the subject, wherein the regimen results in the stabilization or reduction in the cancer stem cell population and a reduction in the bulk size of a tumor and the one or more therapies comprises administering at least one stemness modulating agent. In one embodiment, the regimen achieves a 5%-40%, preferably a 10%-60%, and more preferably a 20 to 99% reduction in the cancer stem cell population, and/or a 5%-40%, preferably a 10%-60%, and more preferably a 20 to 99% reduction in the bulk size of the tumor. In a specific embodiment, the reduction the cancer stem cell population and/or tumor size is achieved after two weeks, a month, two months, three months, four months, six month, nine months, 1 year, 2 years, 3 years, 4 years or more of administration of one or more of the cancer therapies. In a particular embodiment, the stabilization or reduction in the cancer stem cell population is determined by the methods described infra, and the bulk size of the tumor is measured by a method described in infra. In certain embodiments, in accordance with the regimen, the cancer stem cell population and/or the reduction in the tumor size is monitored periodically (e.g., after 2, 5, 10, 20, 30 or more doses of one or more of the therapies or after 2 weeks, 1 month, 2 months, 6 months, 1 year, or more of receiving one or more therapies).

The present disclosure provides methods of preventing, treating and/or managing cancer, the method comprising: (a) administering to a subject in need thereof one or more doses of an effective amount of a therapy; (b) monitoring the cancer stem cell population and the bulk tumor size in or from the subject prior to, during, and/or after the administration of a certain number of doses and prior to the administration of a subsequent dose; and (c) maintaining at least a 5%-40%, preferably a 10%-60%, and more preferably a 20 to 99% reduction in the cancer stem cell population and at least a 5%-40%, preferably a 10%-60%, and more preferably a 20 to 99% reduction in the reduction in the bulk tumor size in the subject by repeating step (a) as necessary. In a specific embodiment, the reduction in the cancer stem cell population is determined by a method described infra, and the reduction in the bulk tumor size is determined by a method known to one of skill in the art, e.g., conventional CT scans, PET scans, bone scans, MRIs or X-ray imaging, among other methods. In some embodiments, the reduction of the cancer stem cell population and the reduction in the bulk tumor size are achieved after 5-30, 10-50, 10-75, 10 to 100, 10 to 150, or 10 to 300 doses of the therapy or after 2 weeks, 1 month, 2 months, 6 months, 1 year, or more of receiving one or more therapies.

In another embodiment, the stemness modulating agent is comprised of an antibody that targets nanog or Oct4. The nanog antibody or Oct4 antibody is conjugated to a radioactive metal ion, such as the alpha-emitters ²¹¹ astatine, ²¹²bismuth, ²¹³bismuth; the beta-emitters ¹³¹iodine, ⁹⁰yttrium, ¹⁷⁷lutetium, ¹⁵³samarium, and ¹⁰⁹palladium; or macrocyclic chelators useful for conjugating radiometal ions, including but not limited to, ¹³¹indium, ¹³¹L, ¹³¹yttrium, ¹³¹holmium, ¹³¹samarium, to polypeptides or any of those listed supra. In certain embodiments, the macrocyclic chelator is 1,4,7,10-tetraazacyclododecane-N,N′,N″,N‴-tetraacetic acid (DOTA), which can be attached to the antibody via a linker molecule. Such linker molecules are commonly known in the art and described in Denardo, et al., 1998, Clin Cancer Res 4(10):2483-90; Peterson, et al., 1999, Bioconjug Chem 10(4):553-7; and Zimmerman, et al., 1999, Nucl Med Biol 26(8):943-50, each incorporated by reference in their entireties.

Antibodies can be produced that bind to nanog. Once at least one successful antibody per group is determined, those antibodies are used to select out subpopulations of cells from tumor samples. This may be accomplished by attaching magnetic particles to antibodies and incubating the conjugated antibodies with cells isolated from the tumor. Following incubation, the cells are run through a magnetic column to separate out cells attached to a magnetic antibody (because of expression of a target surface protein) and non-attached cells will flow through the column. This technique enables purification of individual cell populations within the tumor for further study.

In another embodiment, the disclosure pertains to a therapy involving co-administering a sternness modulating agent and a chemotherapeutic agent. Examples of chemotherapeutic agents that can be co-administered with a stemness modulating agent are provided below:

Examples of chemotherapeutic agents include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthracycline; anthramycin; asparaginase; asperlin; azacitidine (Vidaza); azetepa; azotomycin; batimastat; benzodepa; bicalutamide; bisantrene hydrochloride; bisnafide dimesylate; bisphosphonates (e.g., pamidronate (Aredria), sodium clondronate (Bonefos), zoledronic acid (Zometa), alendronate (Fosamax), etidronate, ibandronate, cimadronate, risedromate, and tiludromate); bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine (Ara-C); dacarbazine; dactinomycin; daunorubicin hydrochloride; decitabine (Dacogen); demethylation agents; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; EphA2 inhibitors; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; herceptin; histone deacetylase inhibitors (HDACs); hydroxyurea; idarubicin hydrochloride; ifosfamide; ilmofosine; imatinib mesylate (Gleevec, Glivec); interleukin II (including recombinant interleukin II, or rIL2), interferon alpha-2a; interferon alpha-2b; interferon alpha-n1; interferon alpha-n3; interferon beta-I a; interferon gamma-I b; iproplatin; irinotecan hydrochloride; lanreotide acetate; lenalidomide (Revlimid); letrozole; leuprolide acetate; liarozole hydrochloride; lometrexol sodium; lomustine; losoxantrone hydrochloride; masoprocol; maytansine; mechlorethamine hydrochloride; anti-CD2 antibodies (e.g., siplizumab (MedImmune Inc.; International Publication No. WO 02/098370, which is incorporated herein by reference in its entirety)); megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxaliplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other examples of chemostherapeutic agents include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, dioxamycin; diphenyl spiromustine; docetaxel; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; HMG CoA reductase inhibitors (e.g., atorvastatin, cerivastatin, fluvastatin, lescol, lupitor, lovastatin, rosuvastatin, and simvastatin); hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; LFA-3TIP (Biogen, Cambridge, Mass.; International Publication No. WO 93/0686 and U.S. Pat. No. 6,162,432); liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maytansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+mycobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; ocreotide; okicenone; oligonucleotides; onapristone; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene therapeutically effective regimens; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; gamma secretase inhibitors, single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; 5-fluorouracil; leucovorin; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; thalidomide; velaresol; veramine; verdins; verteporfin; vinorelbine; vinxaltine; anti-integrin antibodies (e.g., anti-integrin .alpha..sub.v.beta..sub.3 antibodies); vorozole; zanoterone; zeniplatin; zilascorb; and zinostatin stimalamer.

### Identifying and Measuring Cancer Stem Cells

The amount of cancer stem cells can be monitored/assessed using standard techniques known to one of skill in the art. Cancer stem cells can be monitored by, e.g., obtaining a sample, such as a tissue/tumor sample, blood sample or a bone marrow sample, from a subject and detecting cancer stem cells in the sample. The amount of cancer stem cells in a sample (which may be expressed as percentages of, e.g., overall cells or overall cancer cells) can be assessed by detecting the expression of antigens on cancer stem cells. Techniques known to those skilled in the art can be used for measuring these activities. Antigen expression can be assayed, for example, by immunoassays including, but not limited to, western blots, immunohistochemistry, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, immunofluorescence, protein A immunoassays, flow cytometry, and FACS analysis. In such circumstances, the amount of cancer stem cells in a test sample from a subject may be determined by comparing the results to the amount of stem cells in a reference sample (e.g., a sample from a subject who has no detectable cancer) or to a predetermined reference range, or to the patient him/herself at an earlier time point (e.g. prior to, or during therapy).

In a specific embodiment, the cancer stem cell population in a sample from a patient is determined by flow cytometry. This method exploits the differential expression of certain surface markers on cancer stem cells relative to the bulk of the tumor. Labeled antibodies (e.g., fluorescent antibodies) can be used to react with the cells in the sample, and the cells are subsequently sorted by FACS methods. In some embodiments, a combination of cell surface markers are utilized in order to determine the amount of cancer stem cells in the sample. For example, both positive and negative cell sorting may be used to assess the amount of cancer stem cells in the sample. Cancer stem cells for specific tumor types can be determined by assessing the expression of markers on cancer stem cells.

In certain embodiments using flow cytometry of a sample, the Hoechst dye protocol can be used to identify cancer stem cells in tumors. Briefly, two Hoechst dyes of different colors (typically red and blue) are incubated with tumor cells. The cancer stem cells, in comparison with bulk cancer cells, over-express dye efflux pumps on their surface that allow these cells to pump the dye back out of the cell. Bulk tumor cells largely have fewer of these pumps, and are therefore relatively positive for the dye, which can be detected by flow cytometry. Typically a gradient of dye positive (“dye.sup.+”) vs. dye negative (“dye.sup.-”) cells emerges when the entire population of cells is observed. Cancer stem cells are contained in the dye- or dye low (dye.sup.low) population. For an example of the use of the Hoechst dye protocol to characterize a stem cell or cancer stem cell population see Goodell, et al., Blood, 98(4):1166-1173 (2001) and Kondo, et al., Proc Natl Acad Sci USA 101:781-786 (2004). In this way, flow cytometry could be used to measure cancer stem cell amount pre-and post-therapy to assess the change in cancer stem cell amount arising from a given therapy or regimen.

In other embodiments using flow cytometry of a sample, the cells in the sample may be treated with a substrate for aldehyde dehydrogenase that becomes fluorescent when catalyzed by this enzyme. For instance, the sample can be treated with BODIPY® aminoacetaldehyde which is commercially available from StemCell Technologies Inc. as Aldefluor®. Cancer stem cells express high levels of aldehyde dehydrogenase relative to bulk cancer cells and therefore become brightly fluorescent upon reaction with the substrate. The cancer stem cells, which become fluorescent in this type of experiment, can then be detected and counted using a standard flow cytometer. In this way, flow cytometry could be used to measure cancer stem cell amount pre- and post-therapy to assess the change in cancer stem cell amount arising from a given therapy or regimen.

In other embodiments, a sample (e.g., a tumor or normal tissue sample, blood sample or bone marrow sample) obtained from the patient is cultured in in vitro systems to assess the cancer stem cell population or amount of cancer stem cells. For example, tumor samples can be cultured on soft agar, and the amount of cancer stem cells can be correlated to the ability of the sample to generate colonies of cells that can be visually counted. Colony formation is considered a surrogate measure of stem cell content, and thus, can be used to quantitate the amount of cancer stem cells. For instance, with hematological cancers, colony-forming assays include colony forming cell (CFC) assays, long-term culture initiating cell (LTC-IC) assays, and suspension culture initiating cell (SC-IC) assays. In this way, the colony-forming or related assay could be used to measure cancer stem cell amount pre- and post-therapy to assess the change in cancer stem cell amount arising from a given therapy or regimen.

In other embodiments, sphere formation is measured to determine the amount of cancer stem cells in a sample (e.g., cancer stem cells form three-dimensional clusters of cells, called spheres) in appropriate media that is conducive to forming spheres. Spheres can be quantitated to provide a measure of cancer stem cells. See Singh, et al., Cancer Res 63: 5821-5828 (2003). Secondary spheres can also be measured. Secondary spheres are generated when the spheres that form from the patient sample are broken apart, and then allowed to reform. In this way, the sphere-forming assay could be used to measure cancer stem cell amount pre- and post-therapy to assess the change in cancer stem cell amount arising from a given therapy or regimen.

In other embodiments, the amount of cancer stem cells in a sample can be determined with a cobblestone assay. Cancer stem cells from certain hematological cancers form “cobblestone areas” (CAs) when added to a culture containing a monolayer of bone marrow stromal cells. For instance, the amount of cancer stem cells from a leukemia sample can be assessed by this technique. The tumor samples are added to the monolayer of bone marrow stromal cells. The leukemia cancer stem cells, more so than the bulk leukemia cells, have the ability to migrate under the stromal layer and seed the formation of a colony of cells which can be seen visually under phase contrast microscopy in approximately 10-14 days as CAs. The number of CAs in the culture is a reflection of the leukemia cancer stem cell content of the tumor sample, and is considered a surrogate measure of the amount of stem cells capable of engrafting the bone marrow of immunodeficient mice. This assay can also be modified so that the CAs can be quantitated using biochemical labels of proliferating cells instead of manual counting, in order to increase the throughput of the assay. See Chung, et al., Blood 105(1):77-84 (2005). In this way, the cobblestone assay could be used to measure cancer stem cell amount pre- and post-therapy to assess the change in cancer stem cell amount arising from a given therapy or regimen.

In other embodiments, a sample (e.g., a tumor or normal tissue sample, blood sample or bone marrow sample) obtained from the patient is analyzed in in vivo systems to determine the cancer stem cell population or amount of cancer stem cells. In certain embodiments, for example, in vivo engraftment is used to quantitate the amount of cancer stem cells in a sample. In vivo engraftment involves implantation of a human specimen with the readout being the formation of tumors in an animal such as in immunocompromised or immunodeficient mice (such as NOD/SCID mice). Typically, the patient sample is cultured or manipulated in vitro and then injected into the mice. In these assays, mice can be injected with a decreasing amount of cells from patient samples, and the frequency of tumor formation can be plotted vs. the amount of cells injected to determine the amount of cancer stem cells in the sample. Alternatively, the rate of growth of the resulting tumor can be measured, with larger or more rapidly advancing tumors indicating a higher cancer stem cell amount in the patient sample. In this way, an in vivo engraftment model/assay could be used to measure cancer stem cell amount pre- and post-therapy to assess the change in cancer stem cell amount arising from a given therapy or regimen.

In certain in vivo techniques, an imaging agent, or diagnostic moiety, is used which binds to molecules on cancer cells or cancer stem cells, e.g., cancer cell or cancer stem cell surface antigens. For instance, a fluorescent tag, radionuclide, heavy metal, or photon-emitter is attached to an antibody (including an antibody fragment) that binds to a cancer stem cell surface antigen. The medical practitioner can infuse the labeled antibody into the patient either prior to, during, or following treatment, and then the practitioner can place the patient into a total body scanner/developer which can detect the attached label (e.g., fluorescent tag, radionuclide, heavy metal, photon-emitter). The scanner/developer (e.g., CT, MRI, or other scanner, e.g. detector of fluorescent label, that can detect the label) records the presence, amount/quantity, and bodily location of the bound antibody. In this manner, the mapping and quantitation of tag (e.g. fluorescence, radioactivity, etc.) in patterns (i.e., different from patterns of normal stem cells within a tissue) within a tissue or tissues indicates the treatment efficacy within the patient’s body when compared to a reference control such as the same patient at an earlier time point or a patient or healthy individual who has no detectable cancer. For example, a large signal (relative to a reference range or a prior treatment date, or prior to treatment) at a particular location indicates the presence of cancer stem cells. If this signal is increased relative to a prior date it suggests a worsening of the disease and failure of therapy or regimen. Alternatively, a signal decrease indicates that the therapy or regimen has been effective.

In a specific embodiment, the amount of cancer stem cells is detected in vivo in a subject according to a method comprising the steps of: (a) administering to the subject an effective amount of a labeled cancer stem cell marker binding agent that binds to a cell surface marker found on the cancer stem cells, and (b) detecting the labeled agent in the subject following a time interval sufficient to allow the labeled agent to concentrate at sites in the subject where the cancer stem cell surface marker is expressed. In accordance with this embodiment, the cancer stem cell surface marker-binding agent is administered to the subject according to any suitable method in the art, for example, parenterally (such as intravenously), or intraperitoneally. In another embodiment, the cancer stem cell surface marker-binding agent is administered to the subject according to any suitable method in the art, for example, locally (such as directly into the lumen of the bladder), intratumorally or intraperitoneally. In accordance with this embodiment, the effective amount of the agent is the amount which permits the detection of the agent in the subject. This amount will vary according to the particular subject, the label used, and the detection method employed. For example, it is understood in the art that the size of the subject and the imaging system used will determine the amount of labeled agent needed to detect the agent in a subject using an imaging means. In the case of a radiolabeled agent for a human subject, the amount of labeled agent administered is measured in terms of radioactivity, for example from about 5 to 20 millicuries of .sup.99Tc. The time interval following the administration of the labeled agent which is sufficient to allow the labeled agent to concentrate at sites in the subject where the cancer stem cell surface marker is expressed will vary depending on several factors, for example, the type of label used, the mode of administration, and the part of the subject’s body that is imaged. In a particular embodiment, the time interval that is sufficient is 6 to 48 hours, 6 to 24 hours, or 6 to 12 hours. In another embodiment the time interval is 5 to 20 days or 5 to 10 days. The presence of the labeled cancer stem cell surface marker-binding agent can be detected in the subject using imaging means known in the art. In general, the imaging means employed depend upon the type of label used. Skilled artisans will be able to determine the appropriate means for detecting a particular label. Methods and devices that may be used include, but are not limited to, computed tomography (CT), whole body scan such as position emission tomography (PET), magnetic resonance imaging (MRI), and sonography. In a specific embodiment, the cancer stem cell surface marker-binding agent is labeled with a radioisotope and is detected in the patient using a radiation responsive surgical instrument (Thurston, et al., U.S. Pat. No. 5,441,050). In another embodiment, the cancer stem cell surface marker-binding agent is labeled with a fluorescent compound and is detected in the patient using a fluorescence responsive scanning instrument. In another embodiment, the cancer stem cell surface marker-binding agent is labeled with a positron emitting metal and is detected in the patient using positron emission-tomography. In yet another embodiment, the cancer stem cell surface marker-binding agent is labeled with a paramagnetic label and is detected in a patient using magnetic resonance imaging (MRI).

Any in vitro or in vivo (ex vivo) assays known to those skilled in the art that can detect and/or quantify cancer stem cells can be used to monitor cancer stem cells in order to evaluate the prophylactic and/or therapeutic utility of a cancer therapy or regimen disclosed herein for cancer or one or more symptoms thereof; or these assays can be used to assess the prognosis of a patient. The results of these assays then may be used to possibly maintain or alter the cancer therapy or regimen.

The amount of cancer stem cells in a specimen can be compared to a predetermined reference range and/or an earlier amount of cancer stem cells previously determined for the subject (either prior to, or during therapy) in order to gauge the subject’s response to the treatment regimens described herein. In a specific embodiment, a stabilization or reduction in the amount of cancer stem cells relative to a predetermined reference range and/or earlier cancer stem cell amount previously determined for the subject (prior to, during and/or after therapy) indicates that the therapy or regimen was effective and thus possibly an improvement in the subject’s prognosis, whereas an increase relative to the predetermined reference range and/or cancer stem cell amount detected at an earlier time point indicates that the therapy or regimen was ineffective and thus possibly the same or a worsening in the subject’s prognosis. The cancer stem cell amount can be used with other standard measures of cancer to assess the prognosis of the subject and/or efficacy of the therapy or regimen: such as response rate, durability of response, relapse-free survival, disease-free survival, progression-free survival, and overall survival. In certain embodiments, the dosage, frequency and/or duration of administration of a therapy is modified as a result of the determination of the amount or change in relative amount of cancer stem cells at various time points which may include prior to, during, and/or following therapy.

The present disclosure also relates to methods for determining that a cancer therapy or regimen is effective at targeting and/or impairing cancer stem cells by virtue of monitoring cancer stem cells over time and detecting a stabilization or decrease in the amount of cancer stem cells during and/or following the course of the cancer therapy or regimen.

In a certain embodiment, a therapy or regimen may be marketed as an anticancer stem cell therapy or regimen based on the determination that a therapy or regimen is effective at targeting and/or impairing cancer stem cells by virtue of having monitored or detected a stabilization or decrease in the amount of cancer stem cells during therapy.

U.S. Pat. Publications 20070071731; 20060188489; 20060099193; and 20060134789 20080102521 are cited for further discussion of stem cells, and experimental protocols related thereto. U.S. Pat. Pub 20080118518 is cited for use of isolated cancer stem cells and using the knowledge that nanog is differentially expressed therein for screening new potential drug candidates. 20090081214 is cited for further discussion of using a marker, such as the newly discovered nanog, to develop novel cancer therapies. The sequence submitted with the instant application includes the genetic and protein sequence of nanog.

### Antiviral Therapies

Another aspect of the present disclosure pertains to a method of reducing or preventing viral infection comprising delivery of an antiviral knockdown agent to infected or susceptible cells. In certain embodiments, the antiviral knockdown agent is formulated to facilitate intracellular delivery. In a certain aspect of the present disclosure, the antiviral knockdown agent provides for a gene editing selected from CRISPR-Cas9-based gene editing system that disrupts a viral gene.

Yet another aspect of the present disclosure the gene editing system causes an insertion or deletion in an open reading frame of the viral genome. In one embodiment, the open reading frame codes for a spike protein of Sars-Co-2, wherein the insertion disrupts expression of a functioning spike protein.

In some aspects, the antiviral knockdown agent is a therapeutic protein, an antibody, an oligonucleotide-based inhibitor, a gene editing system, or a small molecule drug. In certain aspects, the antibody binds an intracellular viral antigen. In certain aspects, the antibody is a full-length antibody, an scFv, a Fab fragment, a (Fab)2, a diabody, a triabody, or a minibody. In certain aspects, the oligonucleotide based inhibitor is a dsRNA, DNA antisense molecule, siRNA, shRNA, miRNA, or pre-miRNA. In certain aspects, the gene editing system is a CRISPR/Cas system. In certain aspects, the therapeutic protein is a a dominant negative version of a protein that is hyperactive in a cell at the site of the disease or disorder. In some aspects, the small molecule drug is an imaging agent.

The antiviral knockdown agent of the present disclosure can comprise as an active ingredient one or more substances capable of suppressing the expression of the target gene or the activity of the protein encoded by the target gene. The active ingredient is not particularly limited as long as it can suppress the expression of the target gene or the activity of the protein encoded by the target gene. The phrase “suppressing the expression of the target gene or the activity of the protein encoded by the target gene” is synonymous with suppressing the expression or activity of the protein encoded by the target gene. The phrase “suppressing the expression or activity of the protein” refers to any aspect in which the functional expression of the protein is suppressed (or inhibited), and includes, but is not limited to, suppressing the activity (function) of the protein, and suppressing the expression of the protein (e.g. suppression of gene expression, including suppression of the transcription of the gene encoding the protein and suppression of the translation to the protein). Aspects in which the activity of the protein is suppressed include, but are not limited to, inhibition of the binding between a protein receptor and a ligand or an associating molecule, inhibition of the interaction between intracellular proteins, inhibition of the activation of the protein, and inhibition of the enzymatic activity of the protein. The antiviral knockdown agent of the present disclosure may be a drug that inhibits the interaction between the protein encoded by the target gene and a specific gene or a molecule such as a protein. The specific gene or protein or the like may be one which has been revealed to interact with the protein encoded by the target gene, or may be one which will be confirmed to interact with it in the future.

Examples of the active ingredient of the antiviral knockdown agent of the present disclosure include, but are not limited to, inhibitors of the protein encoded by the target gene; antibodies that specifically bind to the protein encoded by the target gene; compounds capable of suppressing the expression of the protein encoded by the target gene; and association inhibitors in cases where the protein acts in association with its target protein(s).

As the above-described inhibitor of the protein encoded by the target gene, any inhibitors for the protein encoded by the target gene which are already known or will be developed in future can be used. Preferably, the above-described inhibitor is an inhibitor specific for the protein encoded by the target gene.

As the above-described antibody that specifically binds to the protein encoded by the target gene, any antibodies capable of inhibiting the function of the protein encoded by the target gene, which are already known or will be developed in future, can be used. For example, antibodies that bind to the active site of the protein encoded by the target gene and inhibit its function are included. Such antibodies may be polyclonal or monoclonal. Both polyclonal antibodies and monoclonal antibodies can be appropriately prepared by methods known to those skilled in the art. When the antibodies are monoclonal, they may be chimeric antibodies, humanized antibodies, or human antibodies prepared by known methods. The antibodies may also be, for example, but are not limited to, complete antibody molecules, antibody fragments, bispecific antibodies, minibodies, domain antibodies, synthetic antibodies (also referred to as “antibody mimetics”), antibody fusions (also referred to as “antibody conjugates”), or fragments thereof. Antibody fragments include Fab fragments, Fd fragments, Fv fragments, dAb fragments, CDR regions, F(ab′)2 fragments, single chain Fvs (ScFvs), minibodies, diabodies, triabodies, and tetrabodies.

The above-described oligonucleotide-based inhibitor for suppressing the expression of the protein encoded by the target gene include, but not limited to, RNA molecules having an RNA interference action (action considered to be based on specifically destroying mRNA derived from the target gene) such as antisense oligonucleotides, shRNAs, siRNAs, and dsRNAs against the target gene or the transcription product thereof; and miRNAs and aptamers considered to be capable of suppressing the translation of the mRNA of the target gene. The antisense oligonucleotide is a single stranded DNA or RNA molecule complementary to the target sequence, and binds to the complementary DNA or RNA to inhibit its expression.

The RNA molecules having an RNA interference action can be appropriately designed by a person skilled in the art by using a known method based on the information about the base sequence of the target gene. The RNA molecule can be prepared by a person skilled in the art according to a known method, and those which are distributed in the market can be obtained and used. As the above-described oligonucleotide based inhibitor capable of suppressing the expression of the protein encoded by the target gene, siRNA, shRNA and miRNA are preferable, and siRNA and shRNA are particularly preferable. The oligonucleotide-based inhibitors capable of suppressing the expression of the protein encoded by the target gene which can be used include, but are not limited to, those having an activity of inhibiting transcription or translation of the gene described above.

The above-described oligonucleotide based inhibitors capable of suppressing the expression of the protein encoded by the target gene is a nucleic acid that binds to a portion of the target gene and suppresses the expression of the protein. The RNA or DNA molecule capable of binding to a portion of the target gene can be introduced into a cell by a method known per se.

The above-described RNA or DNA molecules can be introduced into a cell by using a DNA molecule, such as a vector, capable of expressing these molecules, and the vector can be appropriately prepared by a person skilled in the art by a known method. Specific examples of the vectors include, but are not limited to, adenoviral vectors, lentiviral vectors, and adeno-associated viral vectors. Preferably, the vector is a lentiviral vector.

The antiviral knockdown agent of the present disclosure may be provided as a pharmaceutical composition which contains the said antiviral knockdown agent and is for treatment and/or prevention of diseases associated with viral infection. Examples of the diseases associated with viral infection include coronaviruses. In a specific example, the coronavirus is Sars-CoV-2 or HCoV-229E. The pharmaceutical composition can be formulated according to a known technique. Specific examples of the formulations include, but are not limited to, solid formulations such as tablets, capsules, pills, powders, and granules, and liquid formulations such as solutions, suspensions, emulsions and injections. Depending on the form of the formulation, pharmaceutically acceptable carriers and additives can be added as appropriate. Specific examples of the carriers and additives include, but are not limited to, preservatives, stabilizers, excipients, fillers, binders, wetting agents, flavoring agents, and coloring agents. When the formulation is a liquid formulation, a known pharmaceutically acceptable solvent such as a physiologic saline or a solution having a buffering action can be used.

The dose of the pharmaceutical composition of the present disclosure is not particularly limited as long as it can produce an antiviral effect of the active ingredient, and can be appropriately set by a person skilled in the art. The dose of the active ingredient can be, for example, 0.01 to 1000 mg, preferably 0.05 to 500 mg, more preferably 0.1 to 100 mg per kg body weight of patient per dose.

The method for administering the pharmaceutical composition of the present disclosure is not particularly limited as long as it can produce the antiviral effect, and can be appropriately set by a person skilled in the art. For example, a person skilled in the art can select an administration method needed according to the specific disease state. Specific modes of the administration method include, but are not limited to, injections (such as intravenous, subcutaneous, intramuscular, intraperitoneal, and injection to the affected part), oral, suppository, and transdermal administrations (such as application).

The present disclosure provides a method for treating or preventing a disease associated with viral infection, comprising the step of administering a substance capable of suppressing the expression or activity of the protein encoded by the target gene. The subject to be administered, administration method, dose, etc. are as described above.

The present disclosure further provides a substance capable of suppressing the expression or activity of the protein encoded by the target gene, for use in treatment or prevention of symptoms caused by viral infections.

The present disclosure further provides the use of an antiviral knockdown agent for producing a pharmaceutical composition for treatment and/or prevention of symptoms caused by viral infections.

The antiviral knockdown agent of the present disclosure may be used in combination with other agents effective against viral infection. They may be administered separately during the course of treatment, or may be administered in combination with the antiviral knockdown agent of the present disclosure, for example, in a single dosage form such as a tablet, intravenous solution or capsule. The other agents effective against viral infections include viral growth inhibitors. Viral growth inhibitors preferably used in combination with the antiviral knockdown agent of the present disclosure are reverse transcriptase inhibitors. When the virus is a hepatitis B virus, the viral growth inhibitor used in combination with the antiviral knockdown agent of the present disclosure is an HBV growth inhibitor, including in particular, interferon, peginterferon, lamivudine, adefovir, entecavir, tenofovir, telbivudine, and clevudine, among which entecavir is preferred.

In another embodiment, the present disclosure relates to a screening method for an antiviral knockdown agent, the method comprising selecting a substance capable of suppressing an expression or activity of a protein encoded by a target gene as an antiviral drug from test substances, wherein the target gene is one or more genes selected from the group consisting of ORF4 or spike protein genes of SARs-Cov-2. The screening method of the present disclosure includes the following steps:
(i) determining whether or not the test substance is a substance capable of suppressing the expression or activity of the protein encoded by the target gene; an
(ii) selecting as an active ingredient of the antiviral knockdown agent a test substance determined in the step (i) as a substance capable of suppressing the expression or activity of the protein encoded by the target gene.

By the above-described step (i), whether or not the test substance to be screened is a substance capable of suppressing the expression or activity of the protein encoded by the target gene is determined. Means for determining whether or not the substance is capable of suppressing the expression or activity of the protein encoded by the target gene can be appropriately selected from any means known to those skilled in the art and developed in the future, within the range where the object is achieved, depending on the test substance to be determined and on the expression or activity of the protein encoded by the target gene whose suppression is to be determined. For example, the expression level of the target gene in a cell capable of expressing the target gene, the level of enzymatic activity of the protein encoded by the target gene, the level of activity or function of a protein itself with which the protein encoded by the target gene interacts (associating molecule), or the binding ability or binding amount (association ability or association amount) between the protein encoded by the target gene and the protein (associating molecule) interacting with the protein encoded by the target gene may be used as an indicator. The values of such an indicator may be compared between conditions in which the test substance is absent and present, and when the value of the indicator is decreased in the presence of the test substance compared to the value in the absence of the test substance, the test substance can be determined to be a substance capable of suppressing the expression or activity of the protein encoded by the target gene.

An antiviral knockdown agent of the present disclosure can be administered simultaneously or sequentially with another agent, such as an antiviral, an antibiotic, an anti-inflammatory, or another agent. For example, an antiviral knockdown agent can be administered simultaneously with another agent, such as a known antiviral, an antibiotic or an anti-inflammatory. Simultaneous administration can occur through administration of separate compositions, each containing one or more of an antiviral knockdown agent, a known antiviral, an antibiotic, an anti-inflammatory, or another agent. Simultaneous administration can occur through administration of one composition containing two or more of a antiviral knockdown agent, an antiviral, an antibiotic, an anti-inflammatory, or another agent. An antiviral knockdown agent can be administered sequentially with an antiviral, an antibiotic, an anti- inflammatory, or another agent. For example, an antiviral knockdown agent can be administered before or after administration of an antiviral, an antibiotic, an anti-inflammatory, or another agent.

Anti-inflammatory agents include but are not limited to steroids, e.g., budesonide, nonsteroidal anti-inflammatory agents, e.g., aminosalicylates (e.g., sulfasalazine, mesalamine, olsalazine, and balsalazide), cyclooxygenase inhibitors (COX-2 inhibitors, such as rofecoxib, celecoxib), diclofenac, etodolac, famotidine, fenoprofen, flurbiprofen, ketoprofen, ketorolac, ibuprofen, indomethacin, meclofenamate, mefenamic acid, meloxicam, nambumetone, naproxen, oxaprozin, piroxicam, salsalate, sulindac, tolmetin.

Examples of known antiviral agents that may be co-administered with an antiviral knockdown agent include remdesivir, chloroquine, hydroxychloroquine, atazanavir, daclatasvir, sofosbuvir, ganciclovir, foscamet, cidofovir, indinavir, lopinavir, interferon (e.g. interferon-beta1), ritonavir, AZT, lamivudine and saquinavir.

### Polynucleotides and Expression Products

In the context of the present application, a variant of a polynucleotide sequence is a sequence with at least 70%, preferably at least 80%, most preferably at least 90% sequence identity with the reference sequence. As used herein, a variant of a polypeptide sequence is to be understood to comprise proteins which contain an amino acid sequence identity of at least 70%, at least 80%, at least 90%, at least 95% or typically at least 98% to a reference amino acid sequence. Those skilled in the art will appreciate that amino acids which have corresponding properties, particularly with regard to their charge, hydrophobic character, steric properties, etc. can be substituted in place of a given amino acid.

Sequence identity of nucleotide or amino acid sequences may be determined conventionally by using known software or computer programs such as the BestFit or Gap pairwise comparison programs (GCG Wisconsin Package, Genetics Computer Group, 575 Science Drive, Madison, Wis. 53711). BestFit uses the local homology algorithm of Smith and Waterman, Advances in Applied Mathematics 2: 482-489 (1981), to find the best segment of identity or similarity between two sequences. Gap performs global alignments: all of one sequence with all of another similar sequence using the method of Needleman and Wunsch, J. Mol. Biol. 48:443-453 (1970). When using a sequence alignment program such as BestFit, to determine the degree of sequence identity, the default setting may be used, or an appropriate scoring matrix may be selected to optimize identity, similarity or homology scores. Similarly, when using a program such as BestFit to determine sequence identity, between two different amino acid sequences, the default settings may be used, or an appropriate scoring matrix, such as blosum45 or blosum80, may be selected to optimize identity, similarity or homology scores.

The term “isolated” means separated from its natural environment.

The term “polynucleotide” refers in general to polyribonucleotides and polydeoxyribonucleotides, and can denote an unmodified RNA or DNA or a modified RNA or DNA.

The term “polypeptides” is to be understood to mean peptides or proteins which contain two or more amino acids which are bound via peptide bonds.

Gene product polypeptides of genes to be targeted herein include, but are not limited to, polypeptides corresponding to nanog or Oct4 in case of cancer, or Sars-CoV-2 spike protein or ORF4 in the case viral infection, and variants thereof. See polypeptide sequences provided below:

Nanog nucleic acid sequence (top row) and corresponding amino acid sequence (bottom row)
tttttcctcc tcttcctcta tactaac atg agt gtg gat cca gct tgt ccc caa 54
Met Ser Val Asp Pro Ala Cys Pro Gln
1 5
agc ttg cct tgc ttt gaa gaa tcc gac tgt aaa gaa tct tca cct atg 102
Ser Leu Pro Cys Phe Glu Glu Ser Asp Cys Lys Glu Ser Ser Pro Met
10 15 20 25
cct gtg att tgt ggg cct gaa gaa aac tat cca tcc ttg caa atg tct 150
Pro Val Ile Cys Gly Pro Glu Glu Asn Tyr Pro Ser Leu Gln Met Ser
30 35 40
tct gct gag atg cct cac aca gag act gtc tct cct ctt cct tcc tcc 198
Ser Ala Glu Met Pro His Thr Glu Thr Val Ser Pro Leu Pro Ser Ser
45 50 55
atg gat ctg ctt att cag gac agc cct gat tct tcc acc agt ccc aaa 246
Met Asp Leu Leu Ile Gln Asp Ser Pro Asp Ser Ser Thr Ser Pro Lys
60 65 70
ggc aaa caa ccc act tct gca gag aat agt gtc gca aaa aag gaa gac 294
Gly Lys Gln Pro Thr Ser Ala Glu Asn Ser Val Ala Lys Lys Glu Asp
75 80 85
aag gtc ccg gtc aag aaa cag aag acc aga act gtg ttc tct tcc acc 342
Lys Val Pro Val Lys Lys Gln Lys Thr Arg Thr Val Phe Ser Ser Thr
90 95 100 105
cag ctg tgt gta ctc aat gat aga ttt cag aga cag aaa tac ctc agc 390
Gln Leu Cys Val Leu Asn Asp Arg Phe Gln Arg Gln Lys Tyr Leu Ser
110 115 120
ctc cag cag atg caa gaa ctc tcc aac atc ctg aac ctc agc tac aaa 438
Leu Gln Gln Met Gln Glu Leu Ser Asn Ile Leu Asn Leu Ser Tyr Lys
125 130 135
cag gtg aag acc tgg ttc cag aac cag aga atg aaa tct aag agg tgg 486
Gln Val Lys Thr Trp Phe Gln Asn Gln Arg Met Lys Ser Lys Arg Trp
140 145 150
cag aaa aac aac tgg ccg aag aat agc aat ggt gtg acg cag aag gcc 534
Gln Lys Asn Asn Trp Pro Lys Asn Ser Asn Gly Val Thr Gln Lys Ala
155 160 165
tca gca cct acc tac ccc agc ctc tac tct tcc tac cac cag gga tgc 582
Ser Ala Pro Thr Tyr Pro Ser Leu Tyr Ser Ser Tyr His Gln Gly Cys
170 175 180 185
ctg gtg aac ccg act ggg aac ctt cca atg tgg agc aac cag acc tgg 630
Leu Val Asn Pro Thr Gly Asn Leu Pro Met Trp Ser Asn Gln Thr Trp
190 195 200
aac aat tca acc tgg agc aac cag acc cag aac atc cag tcc tgg agc 678
Asn Asn Ser Thr Trp Ser Asn Gln Thr Gln Asn Ile Gln Ser Trp Ser
205 210 215
aac cac tcc tgg aac act cag acc tgg tgc acc caa tcc tgg aac aat 726
Asn His Ser Trp Asn Thr Gln Thr Trp Cys Thr Gln Ser Trp Asn Asn
220 225 230
cag gcc tgg aac agt ccc ttc tat aac tgt gga gag gaa tct ctg cag 774
Gln Ala Trp Asn Ser Pro Phe Tyr Asn Cys Gly Glu Glu Ser Leu Gln
235 240 245
tcc tgc atg cac ttc cag cca aat tct cct gcc agt gac ttg gag gct 822
Ser Cys Met His Phe Gln Pro Asn Ser Pro Ala Ser Asp Leu Glu Ala
250 255 260 265
gcc ttg gaa gct gct ggg gaa ggc ctt aat gta ata cag cag acc act 870
Ala Leu Glu Ala Ala Gly Glu Gly Leu Asn Val Ile Gln Gln Thr Thr
270 275 280
agg tat ttt agt act cca caa acc atg gat tta ttc cta aac tac tcc 918
Arg Tyr Phe Ser Thr Pro Gln Thr Met Asp Leu Phe Leu Asn Tyr Ser
285 290 295
atg aac atg caa cct gaa gac gtg tga agatgagtga aactgatatt 965
Met Asn Met Gln Pro Glu Asp Val
300 305

actcaatttc agtctggaca ctggctgaat ccttcctctc ccctcctccc atccctcata 1025
ggatttttct tgtttggaaa ccacgtgttc tggtttccat gatgcctatc cagtcaatct 1085
catggagggt ggagtatggt tggagcctaa tcagcgaggt ttcttttttt ttttttccta 1145
ttggatcttc ctggagaaaa tacttttttt tttttttttg agacggagtc ttgctctgtc 1205
gcccaggctg gagtgeagtg gcgcggtctt ggctcactgc aagctccgcc tcccgggttc 1265
acgccattct cctgcctcag cctcccgagc agctgggact acaggcgccc gccacctcgc 1325
ccggctaata ttttgtattt ttagtagaga cagggtttca ctgtgttagc caggatggtc 1385
tcgatctcct gaccttgtga tccgcccgcc tcggcctccc taacagctgg gattacaggc 1445
gtgagccacc gcgccctgcc tagaaaagac attttaataa ccttggctgc taaggacaac 1505
attgatagaa gccgtctctg gctatagata agtagatcta atactagttt ggatatcttt 1565
agggtttaga atctaacctc aagaataaga aatacaagta cgaattggtg atgaagatgt 1625
att 1628

Nanog amino acid sequence
Met Ser Val Asp Pro Ala Cys Pro Gln Ser Leu Pro Cys Phe Glu Glu
1 5 10 15
Ser Asp Cys Lys Glu Ser Ser Pro Met Pro Val Ile Cys Gly Pro Glu
20 25 30
Glu Asn Tyr Pro Ser Leu Gln Met Ser Ser Ala Glu Met Pro His Thr
35 40 45
Glu Thr Val Ser Pro Leu Pro Ser Ser Met Asp Leu Leu Ile Gln Asp
50 55 60
Ser Pro Asp Ser Ser Thr Ser Pro Lys Gly Lys Gln Pro Thr Ser Ala
65 70 75 80
Glu Asn Ser Val Ala Lys Lys Glu Asp Lys Val Pro Val Lys Lys Gln
85 90 95
Lys Thr Arg Thr Val Phe Ser Ser Thr Gln Leu Cys Val Leu Asn Asp
100 105 110
Arg Phe Gln Arg Gln Lys Tyr Leu Ser Leu Gln Gln Met Gln Glu Leu
115 120 125
Ser Asn Ile Leu Asn Leu Ser Tyr Lys Gln Val Lys Thr Trp Phe Gln
130 135 140
Asn Gln Arg Met Lys Ser Lys Arg Trp Gln Lys Asn Asn Trp Pro Lys
145 150 155 160
Asn Ser Asn Gly Val Thr Gln Lys Ala Ser Ala Pro Thr Tyr Pro Ser
165 170 175
Leu Tyr Ser Ser Tyr His Gln Gly Cys Leu Val Asn Pro Thr Gly Asn
180 185 190
Leu Pro Met Trp Ser Asn Gln Thr Trp Asn Asn Ser Thr Trp Ser Asn
195 200 205
Gln Thr Gln Asn Ile Gln Ser Trp Ser Asn His Ser Trp Asn Thr Gln
210 215 220
Thr Trp Cys Thr Gln Ser Trp Asn Asn Gln Ala Trp Asn Ser Pro Phe
225 230 235 240
Tyr Asn Cys Gly Glu Glu Ser Leu Gln Ser Cys Met His Phe Gln Pro
245 250 255
Asn Ser Pro Ala Ser Asp Leu Glu Ala Ala Leu Glu Ala Ala Gly Glu
260 265 270
Gly Leu Asn Val Ile Gln Gln Thr Thr Arg Tyr Phe Ser Thr Pro Gln
275 280 285
Thr Met Asp Leu Phe Leu Asn Tyr Ser Met Asn Met Gln Pro Glu Asp
290 295 300
Val
305

Oct 4 nucleic acid sequence
1 gagtagtccc ttcgcaagcc ctcatttcac caggcccccg gcttggggcg ccttccttcc
61 ccatggcggg acacctggct tcggatttcg ccttctcgcc ccctccaggt ggtggaggtg
121 atgggccagg ggggccggag ccgggctggg ttgatcctcg gacctggcta agcttccaag
181 gccctcctgg agggccagga atcgggccgg gggttgggcc aggctctgag gtgtggggga
241 ttcccccatg ccccccgccg tatgagttct gtggggggat ggcgtactgt gggccccagg
301 ttggagtggg gctagtgccc caaggcggct tggagacctc tcagcctgag ggcgaagcag
361 gagtcggggt ggagagcaac tccgatgggg cctccccgga gccctgcacc gtcacccctg
421 gtgccgtgaa gctggagaag gagaagctgg agcaaaaccc ggaggagtcc caggacatca
481 aagctctgca gaaagaactc gagcaatttg ccaagctcct gaagcagaag aggatcaccc
541 tgggatatac acaggccgat gtggggctca ccctgggggt tctatttggg aaggtattca
601 gccaaacgac catctgccgc tttgaggctc tgcagcttag cttcaagaac atgtgtaagc
661 tgcggccctt gctgcagaag tgggtggagg aagctgacaa caatgaaaat cttcaggaga
721 tatgcaaagc agaaaccctc gtgcaggccc gaaagagaaa gcgaaccagt atcgagaacc
781 gagtgagagg caacctggag aatttgttcc tgcagtgccc gaaacccaca ctgcagcaga
841 tcagccacat cgcccagcag cttgggctcg agaaggatgt ggtccgagtg tggttctgta
901 accggcgcca gaagggcaag cgatcaagca gcgactatgc acaacgagag gattttgagg
961 ctgctgggtc tcctttctca gggggaccag tgtcctttcc tctggcccca gggccccatt
1021 ttggtacccc aggctatggg agccctcact tcactgcact gtactcctcg gtccctttcc
1081 ctgaggggga agcctttccc cctgtctccg tcaccactct gggctctccc atgcattcaa
1141 actgaggtgc ctgcccttct aggaatgggg gacaggggga ggggaggagc tagggaaaga
1201 aaacctggag tttgtgccag ggtttttggg attaagttct tcattcacta aggaaggaat
1261 tgggaacaca aagggtgggg gcaggggagt ttggggcaac tggttggagg gaaggtgaag
1321 ttcaatgatg ctcttgattt taatcccaca tcatgtatca cttttttctt aaataaagaa
1381 gcctgggaca cagtagatag acacactta

Oct4 amino acid sequence
1 maghlasdfa fspppggggd gpggpepgwv dprtwlsfqg ppggpgigpg vgpgsevwgi
61 ppcpppyefc ggmaycgpqv gvglvpqggl etsqpegeag vgvesnsdga spepctvtpg
121 avklekekle qnpeesqdik alqkeleqfa kllkqkritl gytqadvglt lgvlfgkvfs
181 qtticrfeal qlsfknmckl rpllqkwvee adnnenlqei ckaetlvqar krkrtsienr
241 vrgnlenlfl qcpkptlqqi shiaqqlgle kdvvrvwfcn rrqkgkrsss dyaqredfea
301 agspfsggpv sfplapgphf gtpgygsphf talyssvpfp egeafppvsv ttlgspmhsn

Sars-CoV-2 Spike Protein
10 20 30 40 50
MFVFLVLLPL VSSQCVNLTT RTQLPPAYTN SFTRGVYYPD KVFRSSVLHS
60 70 80 90 100
TQDLFLPFFS NVTWFHAIHV SGTNGTKRFD NPVLPFNDGV YFASTEKSNI
110 120 130 140 150
IRGWIFGTTL DSKTQSLLIV NNATNVVIKV CEFQFCNDPF LGVYYHKNNK
160 170 180 190 200
SWMESEFRVY SSANNCTFEY VSQPFLMDLE GKQGNFKNLR EFVFKNIDGY
210 220 230 240 250
FKIYSKHTPI NLVRDLPQGF SALEPLVDLP IGINITRFQT LLALHRSYLT
260 270 280 290 300
PGDSSSGWTA GAAAYYVGYL QPRTFLLKYN ENGTITDAVD CALDPLSETK
310 320 330 340 350
CTLKSFTVEK GIYQTSNFRV QPTESIVRFP NITNLCPFGE VFNATRFASV
360 370 380 390 400
YAWNRKRISN CVADYSVLYN SASFSTFKCY GVSPTKLNDL CFTNVYADSF
410 420 430 440 450
VIRGDEVRQI APGQTGKIAD YNYKLPDDFT GCVIAWNSNN LDSKVGGNYN
460 470 480 490 500
YLYRLFRKSN LKPFERDIST EIYQAGSTPC NGVEGFNCYF PLQSYGFQPT
510 520 530 540 550
NGVGYQPYRV VVLSFELLHA PATVCGPKKS TNLVKNKCVN FNFNGLTGTG
560 570 580 590 600
VLTESNKKFL PFQQFGRDIA DTTDAVRDPQ TLEILDITPC SFGGVSVITP
610 620 630 640 650
GTNTSNQVAV LYQDVNCTEV PVAIHADQLT PTWRVYSTGS NVFQTRAGCL
660 670 680 690 700
IGAEHVNNSY ECDIPIGAGI CASYQTQTNS PRRARSVASQ SIIAYTMSLG
710 720 730 740 750
AENSVAYSNN SIAIPTNFTI SVTTEILPVS MTKTSVDCTM YICGDSTECS
760 770 780 790 800
NLLLQYGSFC TQLNRALTGI AVEQDKNTQE VFAQVKQIYK TPPIKDFGGF
810 820 830 840 850
NFSQILPDPS KPSKRSFIED LLFNKVTLAD AGFIKQYGDC LGDIAARDLI
860 870 880 890 900
CAQKFNGLTV LPPLLTDEMI AQYTSALLAG TITSGWTFGA GAALQIPFAM
910 920 930 940 950
QMAYRFNGIG VTQNVLYENQ KLIANQFNSA IGKIQDSLSS TASALGKLQD
960 970 980 990 1000
VVNQNAQALN TLVKQLSSNF GAISSVLNDI LSRLDKVEAE VQIDRLITGR
1010 1020 1030 1040 1050
LQSLQTYVTQ QLIRAAEIRA SANLAATKMS ECVLGQSKRV DFCGKGYHLM
1060 1070 1080 1090 1100
SFPQSAPHGV VFLHVTYVPA QEKNFTTAPA ICHDGKAHFP REGVFVSNGT
1110 1120 1130 1140 1150
HWFVTQRNFY EPQIITTDNT FVSGNCDVVI GIVNNTVYDP LQPELDSFKE
1160 1170 1180 1190 1200
ELDKYFKNHT SPDVDLGDIS GINASVVNIQ KEIDRLNEVA KNLNESLIDL
1210 1220 1230 1240 1250
QELGKYEQYI KWPWYIWLGF IAGLIAIVMV TIMLCCMTSC CSCLKGCCSC
1260 1270
GSCCKFDEDD SEPVLKGVKL HYT

The terms “stringent conditions” or “stringent hybridization conditions” includes reference to conditions under which a polynucleotide will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing).

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

Specificity is typically the function of post-hybridization washes, the critical factors being the ionic strength and temperature of the final wash solution. For DNA—DNA hybrids, the Tm can be approximated from the equation of Meinkoth and Wahl, Anal. Biochem., 138:267-284 (1984): Tm=81.5oC.+16.6 (log M)+0.41 (% GC)-0.61 (% form)-500/L; where M is the molarity of monovalent cations, % GC is the percentage of guanosine and cytosine nucleotides in the DNA, % form is the percentage of formamide in the hybridization solution, and L is the length of the hybrid in base pairs. The Tm is the temperature (under defined ionic strength and pH) at which 50% of a complementary target sequence hybridizes to a perfectly matched probe. Tm is reduced by about 1° C. for each 1% of mismatching; thus, Tm, hybridization and/or wash conditions can be adjusted to hybridize to sequences of the desired identity. For example, if sequences with approximately 90% identity are sought, the Tm can be decreased 10° C. Generally, stringent conditions are selected to be about 5° C. lower than the thermal melting point (Tm) for the specific sequence and its complement at a defined ionic strength and pH. However, severely stringent conditions can utilize a hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired Tm, those of ordinary skill will understand that variations in the stringency of hybridization and/or wash solutions are inherently described. If the desired degree of mismatching results in a Tm of less than 45° C. (aqueous solution) or 32° C. (formamide solution) it is preferred to increase the SSC concentration so that a higher temperature can be used. An extensive guide to the hybridization of nucleic acids is found in Current Protocols in Molecular Biology, Chapter 2, Ausubel, et al., Eds., Greene Publishing and Wiley-Interscience, New York (2000).

### Downregulating Expression

As used herein, the phrase “gene product” refers to an RNA molecule or a protein, such as nanog or Oct4 (stemness genes) or spike protein or open reading frames of coronaviruses, or RNAs encoding same.

As used herein, the term “downregulating expression” refers to causing, directly or indirectly, reduction in the transcription of a desired gene, reduction in the amount, stability or translatability of transcription products (e.g. RNA) of the gene, and/or reduction in translation of the polypeptide(s) encoded by the desired gene.

It will be appreciated that as well as down-regulating a number of genes, the present disclosure further contemplates using a number of agents to down-regulate the same gene (e.g. a number of dsRNAs each hybridizing to a different segment of the same gene).

Tools which are capable of identifying species-specific sequences may be used for this purpose—e.g. BLASTN and other such computer programs

Downregulating expression of a gene product can be monitored, for example, by direct detection of gene transcripts (for example, by PCR), by detection of polypeptide(s) encoded by the gene RNA (for example, by Western blot or immunoprecipitation), by detection of biological activity of polypeptides encode by the gene (for example, catalytic activity, ligand binding, and the like), or by monitoring changes in tissue (e.g. biopsy sample).

Downregulation of a gene product can be effected on the genomic and/or the transcript level using a variety of agents which interfere with transcription and/or translation (e.g., RNA silencing agents, Ribozyme, DNAzyme and antisense).

According to one embodiment, the agent which down-regulates expression of a gene product is a polynucleotide agent, such as an RNA silencing agent According to this embodiment, the polynucleotide agent is greater than 15 base pairs in length.

As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene RNA sequence. RNA silencing has been observed in many types of organisms, including plants, animals, and fungi.

As used herein, the term “RNA silencing agent” refers to an RNA which is capable of inhibiting or “silencing” the expression of a target gene. In certain embodiments, the RNA silencing agent is capable of preventing complete processing (e.g., the full translation and/or expression) of an mRNA molecule through a post-transcriptional silencing mechanism. RNA silencing agents include noncoding RNA molecules, for example RNA duplexes comprising paired strands, as well as precursor RNAs from which such small noncoding RNAs can be generated. Exemplary RNA silencing agents include dsRNAs such as siRNAs, miRNAs and shRNAs. In one embodiment, the RNA silencing agent is capable of inducing RNA interference. In another embodiment, the RNA silencing agent is capable of mediating translational repression.

RNA interference refers to the process of sequence-specific post-transcriptional gene silencing in animals mediated by short interfering RNAs (siRNAs). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA.

The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

According to one embodiment, the dsRNA is greater than 30 bp. The use of long dsRNAs can provide numerous advantages in that the cell can select the optimal silencing sequence alleviating the need to test numerous siRNAs; long dsRNAs will allow for silencing libraries to have less complexity than would be necessary for siRNAs; and, perhaps most importantly, long dsRNA could prevent viral escape mutations when used as therapeutics.

Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl. Acad. Sci. USA. 2002; 99:1443-1448; Tran N., et al., FEBS Lett. 2004; 573:127-134].

Another method of downregulating a gene product is by introduction of small inhibitory RNAs (siRNAs).

The term “siRNA” refers to small inhibitory RNA duplexes (generally between 18-30 basepairs, between 19 and 25 basepairs) that induce the RNA interference (RNAi) pathway. Typically, siRNAs are chemically synthesized as 21 mers with a central 19 bp duplex region and symmetric 2-base 3′-overhangs on the termini, although it has been recently described that chemically synthesized RNA duplexes of 25-30 base length can have as much as a 100-fold increase in potency compared with 21 mers at the same location. The observed increased potency obtained using longer RNAs in triggering RNAi is theorized to result from providing Dicer with a substrate (27 mer) instead of a product (21 mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned the RNA silencing agent of the present disclosure may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. It will be recognized by one of skill in the art that the resulting single chain oligonucleotide forms a stem-loop or hairpin structure comprising a double-stranded region capable of interacting with the RNAi machinery.

According to another embodiment the RNA silencing agent may be a miRNA. miRNAs are small RNAs made from genes encoding primary transcripts of various sizes. They have been identified in both animals and plants. The primary transcript (termed the “pri-miRNA”) is processed through various nucleolytic steps to a shorter precursor miRNA, or “pre-miRNA.” The pre-miRNA is present in a folded form so that the final (mature) miRNA is present in a duplex, the two strands being referred to as the miRNA (the strand that will eventually basepair with the target) The pre-miRNA is a substrate for a form of dicer that removes the miRNA duplex from the precursor, after which, similarly to siRNAs, the duplex can be taken into the RISC complex. It has been demonstrated that miRNAs can be transgenically expressed and be effective through expression of a precursor form, rather than the entire primary form (Parizotto et al. (2004) Genes & Development 18:2237-2242 and Guo et al. (2005) Plant Cell 17:1376-1386).

Unlike siRNAs, miRNAs bind to transcript sequences with only partial complementarity (Zeng et al., 2002, Molec. Cell 9:1327-1333) and repress translation without affecting steady-state RNA levels (Lee et al., 1993, Cell 75:843-854; Wightman et al., 1993, Cell 75:855-862). Both miRNAs and siRNAs are processed by Dicer and associate with components of the RNA-induced silencing complex (Hutvagner et al., 2001, Science 293:834-838; Grishok et al., 2001, Cell 106: 23-34; Ketting et al., 2001, Genes Dev. 15:2654-2659; Williams et al., 2002, Proc. Natl. Acad. Sci. USA 99:6889-6894; Hammond et al., 2001, Science 293:1146-1150; Mourlatos et al., 2002, Genes Dev. 16:720-728). A recent report (Hutvagner et al., 2002, Sciencexpress 297:2056-2060) hypothesizes that gene regulation through the miRNA pathway versus the siRNA pathway is determined solely by the degree of complementarity to the target transcript. It is speculated that siRNAs with only partial identity to the mRNA target will function in translational repression, similar to an miRNA, rather than triggering RNA degradation.

In one embodiment, synthesis of RNA silencing agents suitable for use with the present disclosure can be effected as follows. The target mRNA (nanog or Oct4 or viral spike protein or other coronavirus gene) is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (wwwdotambiondotcom/techlib/tn/91/912 dothtml).

### Intracellular Delivery

In certain embodiments, the antiviral knockout agent is formulated to facilitate intracellular delivery. A variety of intracellular drug delivery approaches can be implemented including but not limited to, the following (1)-(18):

(1) Cell penetrating agents such as amphiphilic polyproline helix PI 1LRR (such as those described in Li et al., “Cationic Amphiphilic Polyproline Helix PI 1LRR Targets Intracellular Mitochondria,” J. Controlled Release 142:259-266 (2010), which is hereby incorporated by reference in its entirety) or peptide-functionalized quantum dots, such as those described in (Liu et al., “Cell-Penetrating Peptide-Functionalized Quantum Dots for Intracellular Delivery,” J. Nanosci. Nanotechnol. 10:7897-7905 (2010), which is hereby incorporated by reference in its entirety).

(2) Carriers responsive to pH, such as carbonate apatite (Hossain et al., “Carbonate Apatite- Facilitated Intracellularly Delivered siRNA for Efficient Knockdown of Functional Genes,” J. Controlled Release 147: 101-108 (2010), which is hereby incorporated by reference in its entirety).

(3) C2-streptavidin delivery systems, which have been used to facilitate drug delivery to macrophages and T-leukemia cells (such as those described in Fahrer et al., “The C2- Streptavidin Delivery System Promotes the Uptake of Biotinylated Molecules in Macrophages and T-leukemia cells,” Biol. Chem. 391, 1315-1325 (2010), which is hereby incorporated by reference in its entirety).

(4) CH(3)-TDDS drug delivery systems.

(5) Hydrophobic bioactive carriers (such as those described in Imbuluzqueta et al., “Novel Bioactive Hydrophobic Gentamicin Carriers for the Treatment of Intracellular Bacterial Infections,” Acta. Biomater. 7: 1599-1608 (2011), which is hereby incorporated by reference in its entirety).

(6) Exosomes (such as those described in Lakhal et al., “Intranasal Exosomes for Treatment of Neuroinflammation? Prospects and Limitations,” Mol. Ther. 19: 1754-1756 (2011); Zhang et al., “Newly Developed Strategies for Multifunctional Mitochondria-Targeted Agents In Cancer Therapy,” Drug Discovery Today 16: 140-146 (2011), each of which is hereby incorporated by reference in its entirety).

(7) Lipid-based delivery systems (such as those described in Bildstein et al.,“Transmembrane Diffusion of Gemcitabine by a Nanoparticulate Squalenoyl Prodrug: An Original Drug Delivery Pathway,” J. Controlled Release 147: 163-170 (2010); Foged, “siRNA Delivery with Lipid-Based Systems: Promises and Pitfalls,” Curr. Top. Med. Chem. 12:97-107 (2012); Holpuch et al., “Nanoparticles for Local Drug Delivery to the Oral Mucosa: Proof of Principle Studies,” Pharm. Res. 27: 1224-1236 (2010); Kapoor et al., “Physicochemical Characterization Techniques for Lipid Based Delivery Systems for siRNA,” Int. J. of Pharm. 427, 35-57 (2012), each of which is hereby incorporated by reference in its entirety), including microtubules, such as those described in (Kolachala et al., “The Use of Lipid Microtubes as a Novel Slow-Release Delivery System for Laryngeal Injection,” The Laryngoscope 121 : 1237- 1243 (2011), which is hereby incorporated by reference in its entirety).

(8) Liposome or liposome-based delivery systems.

(9) Micelles, including disulfide cross-linked micelles, such as those described in (Li et al., “Delivery of Intracellular- Acting Biologies in Pro-Apoptotic Therapies,” Curr. Pharm. Des. 17:293-319 (2011), which is hereby incorporated by reference in its entirety). Carriers with disulfide bonds can be formulated so that one or more disulfide bonds link to the to the antiviral knockout agent, such as oligonucleotide based inhibitors. A variety of micelles have been described, such as phospholipid-polyaspartamide micelles for pulmonary delivery.

(10) Microparticles, such as those described in (Ateh et al., “The Intracellular Uptake of CD95 Modified Paclitaxel-Loaded Poly(Lactic-Co-Glycolic Acid) Microparticles,” Biomater. 32:8538-8547 (2011), which is hereby incorporated by reference in its entirety).

(11) Molecular carriers, such as those described in (Hettiarachchi et al., “Toxicology and Drug Delivery by Cucurbit[n]uril Type Molecular Containers,” PloS One 5:e10514 (2010), which is hereby incorporated by reference in its entirety).

(12) Nanoparticles referred to as ‘nanocarriers’, such as those described in (Gu et al., “Tailoring Nanocarriers for Intracellular Protein Delivery,” Chem. Soc. Rev. 40:3638-3655 (2011), which is hereby incorporated by reference in its entirety), some of which have been formulated for delivery of agents to HIV infected cells, such as those described in (Gunaseelan et al., “Surface Modifications of Nanocarriers for Effective Intracellular Delivery of Anti-HIV Drugs,” Adv. Drug Delivery Rev. 62:518-531 (2010), which is hereby incorporated by reference in its entirety).

(13) Nanoscopic multi-variant carriers.

(14) Nanogels (such as those described in Zhan et al., “Acid-Activatable Prodrug Nanogels for Efficient Intracellular Doxorubicin Release,” Biomacromolecules 12:3612-3620 (2011) and Zhang et al., “Folate-Mediated poly(3-hydroxybutyrate-co-3-hydroxyoctanoate) Nanoparticles for Targeting Drug Delivery,” Eur. J. Pharm. Biopharm. 76: 10-16 (2010), each of which is hereby incorporated by reference in its entirety).

(15) Hybrid nanocarrier systems, which consist of components of two or more particulate delivery systems (such as those described in Pittella et al., “Enhanced Endosomal Escape of siRNA-Incorporating Hybrid Nanoparticles from Calcium Phosphate and PEG-Block Charge- Conversional Polymer for Efficient Gene Knockdown With Negligible Cytotoxicity,” Biomater. 32:3106-3114 (2011), which is hereby incorporated by reference in its entirety). Copolymeric micelle nanocarrier (such as those described in Chen et al., “pH and Reduction Dual-Sensitive Copolymeric Micelles for Intracellular Doxorubicin Delivery,” Biomacromolecules 12:3601- 3611 (2011), which is hereby incorporated by reference in its entirety); liposomal nanocarriers, (such as those described in (Kang et al., “Design of a Pep-1 Peptide-Modified Liposomal Nanocarrier System for Intracellular Drug Delivery: Conformational Characterization and Cellular Uptake Evaluation,” J. of Drug Targeting 19:497-505 (2011), which is hereby incorporated by reference in its entirety). (16) Nanoparticles can be constructed with a variety of nanomaterials (such as those described in Adeli et al., “Synthesis of New Hybrid Nanomaterials: Promising Systems for Cancer Therapy,” Nanomed. Nanotechnol. Biol. Med. 7:806-817 (2011); Al-Jamal et al., “Enhanced Cellular Internalization and Gene Silencing with a Series of Cationic Dendron- Multiwalled Carbon Nanotube:siRNA Complexes,” FASEB J24:4354-4365 (2010); Bulut et al., “Slow Release and Delivery of Antisense Oligonucleotide Drug by Self-Assembled Peptide Amphiphile Nanofibers,” Biomacromolecules 12:3007-3014 (2011), each of which is hereby incorporated by reference in its entirety).

(17) Peptide-based drug delivery systems, which include a variety of cell penetrating peptides and including (but not limited to) TAT-based delivery systems (such as those described in Johnson et al., “Therapeutic Applications of Cell-Penetrating Peptides,” Methods Mol. Biol. 683 :535-551 (2011), which is hereby incorporated by reference in its entirety). Such peptides can be chemically linked to an antiviral knockout agent.

(18) Polymers or copolymer-based delivery systems, such as those described in (Edinger et al., “Bioresponsive Polymers for the Delivery of Therapeutic Nucleic Acids,” Wiley Interdiscip. Rev. Nanomed. and Nanobiotechnol. 3 :33-46 (2011), which is hereby incorporated by reference in its entirety).

### Exosomes

According to certain embodiments, stemness modulating agents or antiviral knockdown agents are loaded into and delivered into exosomes or exosome like vesicles. Exosomes may be produced and loaded with stemness modulating agents or antiviral knockdown agents according to techniques known in the art. See for example US20190093105 and US20190338314. Examples of exosome-producing cells, in approximate order of exosome production from least to most in standard cell culture conditions, may include (but are not limited to):
glioblastoma cell line U251-MG;
epithelial and fibroblast cells like HeLa, MDA-MB-231, and HCT-116 cells (produce moderate amounts of exosomes); and
neurons, immune and blood cells (including dendritic cells, macrophages, T cells, B cells, reticulocytes), mesenchymal stem cells, and embryonic stem cells (produce abundant exosomes).

In certain non-limiting embodiments, the exosome-producing cells may be human cells. Exosomes produced by human cells may have reduced immunogenicity as compared to exosomes from mouse cells when introduced into human patients, which may be due to decreased differences in histocompatibility complexes (Bach, 1987, N Engl J Med, 317:489).

In another non-limiting embodiment, the exosome-producing cells may be embryonic stem cell (ESC) clone H1 or H9 cells, or a mesenchymal stem cell (MSC).

In another non-limiting embodiment, an exosome-producing cell may be an induced pluripotent stem cell, such as an induced pluripotent stem cell derived from a patient to be treated.

In certain non-limiting embodiments, the exosome-producing cells may be cultured in serum-free media, or in serum media which has been previously treated or processed to remove or reduce exosomal content (i.e. exosome-depleted serum media), while producing exosomes or exosome-like vesicles, so as to prevent or reduce contamination of produced exosomes with exosomes typically present in typical serum-containing media.

In certain non-limiting embodiments involving cells which require serum-containing media for growth, it may also be possible to remove the serum media and culture the cells temporarily in serum-free media during production/harvest of produced exosomes being released into the serum-free media. In certain cases, however, abrupt removal of serum media may decrease exosome production in certain cells.

Generally speaking, exosomes are typically 40-150 nm vesicles released by a variety of cell types. Exosomes may be composed of a lipid bilayer and a luminal space containing a variety of proteins, RNAs and other molecules derived from the cytoplasm of the exosome-producing cell. Both the membrane and lumen contents of exosomes may be selectively enriched in subpopulations of lipids, proteins and RNA from the exosome-producing cell. The exosome membrane is frequently, but not necessarily, enriched in lipids including cholesterol and sphingomyelin and contain less phosphatidycholine. The membrane of exosomes may be enriched in particular proteins derived from the plasma membrane of cells such as tetraspanins (e.g. CD63, CD81 CD9), PrP and MHC class I, II. The exosome lumen may be enriched in proteins such as Flotillin1 and 2, annexin 1 and 2, heat shock proteins, Alix and Tsg101. Exosomes are frequently enriched in miR-451 or prc-miR-451.

It will be understood that exosomes as described herein may, in certain non-limiting embodiments, also encompass exosome-like vesicles. The person of skill in the art will recognize that references to exosomes herein may include other suitable exosome-like vesicles which may vary somewhat from typical exosomes, but are still functionally and/or structurally similar or related.

It will also be understood that exosome-producing cells as described herein may, in certain non-limiting embodiments, also encompass exosome-like vesicle-producing cells. The person of skill in the art will recognize that references to exosome-producing cells herein may include other suitable exosome-like vesicle-producing cells which produce exosome-like vesicles which may vary somewhat from typical exosomes but are still functionally and/or structurally similar or related.

As will be understood by the person of skill in the art, exosomes as described herein may also include, in certain non-limiting embodiments, other suitable exosome-like vesicles between 50-150 nm (which contain exosomal markers), and/or larger exosome-like vesicles of 100-600 nm.

### Liposomes

Exemplary formulations suitable as vehicles or carriers for delivery of a polypeptide, pharmaceutical composition, nucleic acid, vector, composition, or host cell described herein, include microemulsions, monolayers, micelles, bilayers, vesicles or lipid particles. These formulations provide a biocompatible and biodegradable delivery system for a polypeptide, pharmaceutical composition, nucleic acid, vector, composition, or host cell described herein,.

Liposomes provide an example of lipid particles, which are composed of amphiphilic lipids arranged in a spherical bilayer or bilayers. Liposomes are unilamellar or multilamellar vesicles which have a membrane formed from a lipophilic material and an aqueous interior. The aqueous portion comprises the a polypeptide, pharmaceutical composition, nucleic acid, vector, composition, or host cell described herein, to be delivered. Cationic liposomes possess the advantage of being able to fuse to the cell wall. Non-cationic liposomes, although not able to fuse as efficiently with the cell wall, are taken up by macrophages in vivo.

Liposomes have several advantages including a small diameter; biocompatibility and biodegradability; ability to incorporate a wide range of contents, e.g., water and lipid soluble drugs. Liposomes can protect encapsulated drugs in their internal compartments from metabolism and degradation (Rosoff, in Pharmaceutical Dosage Lorms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes.

Liposomes fall into two broad classes. Cationic liposomes are positively charged liposomes which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985).

Liposomes which are pH-sensitive or negatively-charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally- derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Exemplary non-ionic liposomal systems suitable for delivery of drugs to the skin include systems comprising non-ionic surfactant and cholesterol. Non-ionic liposomal formulations comprising Novasome™ I (glyceryl dilaurate/cholesterol/polyoxyethylene-10-stearyl ether) and Novasome™ II (glyceryl distearate/cholesterol/polyoxyethylene- 10-stearyl ether) were used to deliver cyclosporin-A into the dermis of mouse skin. Results indicated that such non-ionic liposomal systems were effective in facilitating the deposition of cyclosporin-A into different layers of the skin (Hu et al. S.T.P.Pharma. Sci., 1994, 4, 6, 466).

Liposomes can be sterically stabilized to include one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside GMI, or (B) is derivatized with one or more hydrophilic polymers, such as a polyethylene glycol (PEG) moiety (Allen et al., FEBS Letters, 1987, 223, 42; Wu et al., Cancer Research, 1993, 53, 3765). Long-circulating, e.g., stealth, liposomes can also be employed. Such liposomes are generally described in U.S. Pat. No. 5,013,556. The compounds disclosed herein can also be administered by controlled release means and/or delivery devices such as those described in U.S. Pat. Nos. 3,845,770; 3,916,899; 3,536,809; 3,598,123; and 4,008,719.

Various liposomes comprising one or more glycolipids are known in the art.

Papahadjopoulos et al. (Ann. N.Y. Acad. Sci., 1987, 507, 64) reported the ability of

monosialoganglioside GMI, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and WO 88/04924, both to Allen et al., disclose liposomes comprising (1) sphingomyelin and (2) the ganglioside GMI or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al).

Liposomes comprising lipids can be derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2Cm_{5G}, that contains a PEG moiety. Ilium et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B 1 and WO 90/04384 to Fisher. Liposome compositions containing 1- 20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.) Liposomes comprising PEG-modified ceramide lipids are described in WO 96/10391 (Choi et al). U.S. Pat. No. 5,540,935 (Miyazaki el al.) and U.S. Pat. No. 5,556,948 (Tagawa et al.) describe PEG-containing liposomes that can be further derivatized with functional moieties on their surfaces.

A number of liposomes comprising nucleic acids are known in the art. WO 96/40062 to Thierry et al. discloses methods for encapsulating high molecular weight nucleic acids in liposomes. U.S. Pat. No. 5,264,221 to Tagawa et al. discloses protein-bonded liposomes. U.S. Pat. No. 5,665,710 to Rahman et al. describes certain methods of encapsulating oligodeoxynucleotides in liposomes.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations. The use of surfactants in drug products, formulations and in emulsions has been reviewed (Rieger, in Pharmaceutical Dosage Forms, Marcel Dekker, Inc., New York, N.Y., 1988, p. 285).

Another example of delivery vehicles include nano structured lipid carriers (NLCs), which are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid- polymer nanoparticles (PLNs), combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. For a review, see, e.g., Li et al. 2017, Nanomaterials 7, 122; doi:10.3390/nano7060122.

In some embodiments, a nucleic acid, vector, or composition described herein can be encapsulated in a lipid formulation, e.g., to form a nucleic acid-lipid particle. Nucleic acid lipid particles typically contain a cationic lipid, a non-cationic lipid, and a lipid that prevents aggregation of the particle (e.g., a PEG-lipid conjugate). These particles are useful for systemic applications, as they exhibit extended circulation lifetimes following intravenous (i.v.) injection and accumulate at distal sites (e.g., sites physically separated from the administration site).

Particles which include an encapsulated condensing agent-nucleic acid complex as set forth in PCT Publication No. WO 00/03683. The particles typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles of the present invention are resistant in aqueous solution to degradation with a nuclease. Nucleic acid- lipid particles and their method of preparation are disclosed in, e.g., U.S. Pat. Nos. 5,976,567; 5,981,501; 6,534,484; 6,586,410; 6,815,432; and PCT Publication No. WO 96/40964.

In one embodiment, the lipid to drug ratio (mass/mass ratio) (e.g., lipid to dsRNA ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1.

The cationic lipid may be, for example, N,N-dioleyl-N,N-dimethylammonium chloride (DODAC), N,N-distearyl-N,N-dimethylammonium bromide (DDAB), N-(I -(2,3-dioleoyloxy)propyl)-N,N,N-trimethylammonium chloride (DOTAP), N-(I -(2,3-dioleyloxy)propyl)-N,N,N-trimethylammonium chloride (DOTMA), N,N-dimethyl-2,3-dioleyloxy)propylamine (DODMA), 1 ,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1 ,2-Dilinoleyoxy-3-(dimethylamino)acetoxypropane (DLin-DAC), 1,2-Dilinoleyoxy-3-morpholinopropane (DLin- MA), 1,2-Dilinoleoyl-3-dimethylaminopropane (DLinDAP), 1,2-Dilinoleylthio-3-dimethylaminopropane (DLin-S-DMA), l-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N- methylpiperazino)propane (DLin-MPZ), or 3-(N,N-Dilinoleylamino)- 1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DM A), 1,2-Dilinolenyloxy-N,N-dimethylaminopropane (DLinDMA), 2,2-Dilinoleyl-4-dimethylaminomethyl- [1,3] -dioxolane (DLin-K-DMA) or analogs thereof, (3aR,5s,6aS)-N,N-dimethyl-2,2-di((9Z,12Z)-octadeca-9,12- dienyl)tetrahydro-3aH-cyclopenta[d][l,3]dioxol-5-amine, (6Z,9Z,28Z,31Z)-heptatriaconta- 6,9,28,3 1-tetraen-19-yl 4-(dimethylamino)butanoate (MC3), 1,1′-(2-(4-(2-((2-(bis(2- hydroxydodecyl)amino)ethyl)(2-hydroxydodecyl)amino)ethyl)piperazin-1-yl)ethylazanediyl)didodecan-2-ol (Tech Gl), or a mixture thereof. The cationic lipid may comprise from about 20 mol % to about 50 mol % or about 40 mol % of the total lipid present in the particle.

In one embodiment, the lipid particle includes 40% 2, 2-Dilinoleyl-4-dimethylaminoethyl-[l,3]-dioxolane: 10% DSPC: 40% Cholesterol: 10% PEG-C-DOMG (mole percent) with a particle size of 63.0 ± 20 nm and a 0.027 siRNA/Lipid Ratio.

The non-cationic lipid may be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl- phosphatidylethanolamine 4-(N-maleimidomethyl)-cyclohexane-l-carboxylate (DOPE- mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1 -trans PE, 1 -stearoyl-2-oleoyl-phosphatidyethanolamine (SOPE), cholesterol, or a mixture thereof. The non-cationic lipid may be from about 5 mol % to about 90 mol %, about 10 mol %, or about 58 mol % if cholesterol is included, of the total lipid present in the particle.

The conjugated lipid that inhibits aggregation of particles may be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate may be, for example, a PEG-dilauryloxypropyl (CC), a PEG- dimyristyloxypropyl (C14), a PEG-dipalmityloxypropyl (Ci₆), or a PEG- distearyloxypropyl (C]s). The conjugated lipid that prevents aggregation of particles may be from 0 mol % to about 20 mol % or about 2 mol % of the total lipid present in the particle.

In some embodiments, the nucleic acid-lipid particle further includes cholesterol at, e.g., about 10 mol % to about 60 mol % or about 48 mol % of the total lipid present in the particle.

In one embodiment, the formulations is an MC3 comprising formulations are described, e.g., in International Application No. PCT/US 10/28224, filed Jun. 10, 2010, which is hereby incorporated by reference. The synthesis and structure of MC3 containing formulations is described in, e.g., pages 114-119 of WO 2013/155204, incorporated by reference. In some embodiments, the MC3 formulation comprises a preparation of DLin-M-C3-DMA(_{.<?.,}(6Z,9Z,28Z,31Z)-heptatriaconta-6,9,28,31-tetraen-19-yl4-(dimethylamino)butanoate).

In some embodiments, an antiviral knockdown agent described herein may be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer.

Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi: 10.1155/2011/469679 for review).

Vesicles can be made from several different types of lipids; however, phospholipids are most commonly used to generate liposomes as drug carriers. Vesicles may comprise without limitation DOTMA, DOTAP, DOTIM, DDAB, alone or together with cholesterol to yield DOTMA and cholesterol, DOTAP and cholesterol, DOTIM and cholesterol, and DDAB and cholesterol. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi: 10.1155/2011/469679 for review). Extruded lipids can be prepared by extruding through filters of decreasing size, as described in Templeton et ah, Nature Biotech, 15:647-652, 1997, the teachings of which relating to extruded lipid preparation are incorporated herein by reference.

Lipid nanoparticles (LNPs) are another example of a carrier that provides a biocompatible and biodegradable delivery system for the pharmaceutical compositions described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

### Gene Editing

Thus, as used herein, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (“guide RNA” or “gRNA” in the context of an endogenous CRISPR system), or other sequences and transcripts from a CRISPR locus. One or more tracr-mate sequences operably linked to a guide sequence (e.g., direct repeat-spacer-direct repeat) can also be referred to as “pre-crRNA” (pre- CRISPR RNA) before processing or crRNA after processing by a nuclease.

In some embodiments, a tracrRNA and crRNA are linked and form a chimeric crRNA-tracrRNA hybrid where a mature crRNA is fused to a partial tracrRNA via a synthetic stem loop to mimic the natural crRNA:tracrRNA duplex as described in Cong, Science, 15:339(6121):819-823 (2013) and Jinek, et al, Science, 337(6096):816-21 (2012)). A single fused crRNA-tracrRNA construct can also be referred to as a guide RNA or gRNA (or single-guide RNA (sgRNA)). Within a sgRNA, the crRNA portion can be identified as the ‘target sequence’ and the tracrRNA is often referred to as the ‘scaffold’ RNA (scRNA).

There are many resources available for helping practitioners determine suitable target sites once a desired DNA target sequence is identified. For example, numerous public resources, including a bioinformatically generated list of about 190,000 potential sgRNAs, targeting more than 40% of human exons, are available to aid practitioners in selecting target sites and designing the associated sgRNA to affect a nick or double strand break at the site. See also, crispr.u-psud.fr, a tool designed to help scientists find CRISPR targeting sites in a wide range of species and generate the appropriate crRNA sequences.

While the specifics can be varied in different engineered CRISPR systems, the overall methodology is similar. For example, a practitioner interested in using CRISPR technology to target a DNA sequence can insert a short DNA fragment containing the target sequence into a guide RNA expression plasmid. The sgRNA expression plasmid therefore contains the target sequence (about 20 nucleotides), a form of the tracrRNA sequence (i.e., the scRNA) as well as a suitable promoter and necessary elements for proper processing in eukaryotic cells. Such vectors are commercially available (see, for example, Addgene). Many of the systems rely on custom, complementary oligos that are annealed to form a double stranded DNA and then cloned into the sgRNA expression plasmid. Co-expression of the sgRNA and the appropriate Cas enzyme from the same or separate plasmids in transfected cells results in a single or double strand break (depending of the activity of the Cas enzyme) at the desired target site.

Typically, as used in accordance with the present disclosure, the CRISPR complex is introduced into a cell, and creates a break (e.g., a single or a double strand break) in the target DNA sequence. For example, the method can be used to cleave a target viral gene of a DNA virus that has infected a cell. The break created by the CRISPR complex can be repaired by repair processes such as the error prone nonhomologous end joining (NHEJ) pathway or the high fidelity homology-directed repair (HDR). During these repair processes, an exogenous polynucleotide template can be introduced into the genome sequence. In some methods, the HDR process is used to modify a genome sequence. For example, an exogenous polynucleotide template comprising a sequence to be integrated flanked by an upstream sequence and a downstream sequence is introduced into a cell. The upstream and downstream sequences share sequence similarity with either side of the site of integration in the DNA viral genome. Where desired, a donor polynucleotide can be DNA, e.g., a plasmid DNA (pDNA), a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a exosome, liposome or poloxamer. Thus, the modifications of the target DNA due to NHEJ and/or homology-directed repair can be used to induce transgene insertion, nucleotide deletion, gene disruption, gene mutation, etc.

Accordingly, the present invention provides an expression system for delivering a CRISPR system to cells harboring DNA viral DNA, such that expression of the elements of the CRISPR system direct formation of a CRISPR complex at the target site, which leads to inactivation of the target sequence of the viral DNA.

### Vectors

In other embodiments, the antiviral knockdown agent or stemness modulating agents are delivery via vectors.

As used herein, a “vector” is a tool that allows or facilitates the transfer of an entity from one environment to another. It is a plasmid, phage, or cosmid, into which another DNA segment may be inserted so as to bring about the replication of the inserted segment in the appropriate prokaryotic or eukaryotic cell. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. The vector can be made synthetically using appropriate primers and a high fidelity proof reading DNA polymerase. Another type of vector is a “viral vector,” wherein virally-derived DNA or RNA sequences are present in the vector for packaging into a virus (e.g., retroviruses, replication defective retroviruses, adenoviruses, replication defective adenoviruses, and adeno-associated viruses, AAVs). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively -linked to the nucleic acid sequence to be expressed. Within a recombinant expression vector, “operably linked” is intended to mean that the nucleotide sequence of interest is linked to the regulatory element(s) in a manner that allows for expression (e.g., transcription and translation) of the nucleotide sequence in a host cell when the vector is introduced into the host cell.

The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences).

Accordingly, in one aspect, the invention provides a therapeutic expression plasmid for knocking down a viral gene thereby treating or preventing the viral infection in a subject (e.g. coronavirus infection) and mitigating the risk of developing viral symptoms. The expression system may comprise a gene editing expression plasmid that includes at least one a promoter, at least one enhancer, a 5′ untranslated region (5′-UTR), a nuclease spaced apart from the 5′-UTR by a spacer or intron, and a 3′ untranslated region (3′-UTR), all of which will be explained in detail below.

As used herein, a “promoter” is defined as a regulatory DNA sequence generally located upstream of a gene that mediates the initiation of transcription by directing RNA polymerase to bind to DNA and initiating RNA synthesis. A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular compound or protein), it may be a spatially restricted promoter (i. e., transcriptional control element, enhancer, etc.) (e.g., tissue specific promoter, cell type specific promoter, etc.), and it may be a temporally restricted promoter (i.e., the promoter is in the “ON” state or “OFF” state during specific stages of embryonic development or during specific stages of a biological process.

A tissue-specific promoter may direct expression primarily in a desired tissue of interest, such as a specific organ (e.g., lung cells or vascular cells), or particular cell types (e.g., epithelial cells or endothelial cells). Thus, the plasmid of the invention includes a promoter that is selectively active in such cells that will transcribe CRISPR RNA only in cells where viral replication occurs. In various embodiments, the plasmid may express the gRNAs under a RNA polymerase II (pol II) promoter along with the nuclease. An exemplary promoter useful in the plasmid of the invention includes, but is not limited to, the elongation factor- 1 alpha (EF-la) promoter. Alternatively, the gRNAs can be expressed using a RNA polymerase III (pol III) promoter, such as the U6 promoter that is commonly used for driving small hairpin RNA (shRNA) expression. Exemplary RNA pol III promoters useful in the invention include, but are not limited to, U6 promoter, 7SK promoter, and HI promoter.

### EXAMPLES

### Example 1: Knockdown of Nanog and Oct4 Increases Sensitivity of Cancer Stem Cells to Chemotherapeutic Agents

### Methods

### Silencing NANOG and OCT4 Expression of CD133⁺ GBM

shRNA specifically designed to silence NANOG/P8 and OCT4 expression were delivered to cells via lentiviral transduction. Lentiviral particles were created with shRNA plasmids (sh-NANOG & sh-OCT4) and third generation plasmids pLP-VSVG, pLP1 and pLP2. Cancer stem cells (CD133⁺ GBMs) were transduced with lentiviral particles creating two groups, CD133⁺ GBM with NANOG expression silenced and CD133⁺ GBM with OCT4 expression silenced. In cell culture, at the time of transduction, 4 µg/mL of polybrene (MilliporeSigma, Burlington, MA, USA) was used to reduce the cell surface charge and increase the adherence of viral particles. The following morning, cells were transferred to a conical tube, spun down at 300 g for 3 min, and supernatant was removed and discarded. Cell pellet was resuspended in new culture medium and allowed to rest for two days. Transduction efficacy was measured by using mCherry expressions with fluorescent microscopy (ZEISS Observer. A1), Cells were transferred into HNSC media with 400 µg/mL of Geneticin (Life technology, Carlsbad, CA, USA) for selection of cells with shRNA. Culture media was changed every two to three days for a week. Geneticin concentration was lowered to 200 µg/mL for maintenance from then on.

Currently we are using exosome to deliver shRNA instead of virus and getting the same results.

### TMZ Viability Assay

Working stocks of Temozolomide (TMZ) with concentrations of 0.1 µM-1 mM was created by performing a ⅒ serial dilution using cell culture medium from a 0.1 M TMZ solution in 100% DMSO. Negative Controls 1% and 0.1% DMSO solutions were prepared from a 100% DMSO stock. Silenced and non-Silenced CD133+ GBM cells were dissociated to single-cell suspension using StemPro Accutase and seeded into a 96 round bottom suspension plate at 5,000 and 100,000 cells per well. Appropriate amounts of media and cells filled wells up to 180 µL. 20 µL of testing solutions were then added creating a ⅒ dilution in each well. Triplicates were made for each condition/treatment/sample for this experiment and the 96-well plate was incubated at 37° C., 5% CO2 for 24 hours. After 24 hours, cell viability was measured using LIVE/DEAD Viability/Cytotoxicity kit for mammalian cells (Invitrogen, Carlsbad, CA, USA) and read with the EnVision 2104 Multilabel Reader (PerkinElmer, Waltham, MA, USA).

### Statistical Analysis and Graphs

Statistical analysis was done using Two-way ANOVA analysis using the post-hoc analysis, Fisher least significant difference test.

### Results

Cancer stem cells were treated with the different concentrations of TMZ for 24 hours; the following day cell viability was measured using the live/dead assay kit contains calcein AM and ethidium homodimer-1 (EthD-1). Calcein can stain live cells while EthD-1 stains dead cells. Once EthD-1 penetrates the membrane of dead cells, it emits a strong fluorescent signal after interacting with nucleic acids. The amount of fluorescence is correlated with cell death in this result. After treatment with 10, 100, and 1000 µM TMZ non-NANOG or OCT4 silenced CD133+ GBM Cells showed TMZ concentration dependent cell death though it was very minimum. Little to no cell death was observed as is apparent with cells treated with just 1% DMSO in the same over 24 hours period. Significantly increased cell deathwas observed with NANOG or OCT4 silenced cells as compared to the non-silenced cells when given the same concentration of TMZ. Interestingly, cells treated with 1% DMSO also experienced significant cell death indicating that increased sensitivity to DMSO once NANOG or OCT4 expression was inhibited. See FIGS. 1 and 2.

This result indicates that silencing either NANOG or OCT4 in cancer stem cells makes them more susceptible to any potentially toxic reagent.

We further tested whether GBM CSCs would be affected by TMZ treatment at lower concentrations. Again, cancer stem cells treated with shRNA for NANOG and Oct4 significantly increased cell death than non-silenced GBM CSCs, even at lower TMZ concentrations. Also, cells were much more vulnerable to the much lower concentration of DMSO when NANOG or OCT4 is silenced. This result indicates that we can even lower the concentration of TMZ, which does not show the side effect of this drug. Since these stemness genes are not expressed in any healthy cells we do not expect any side effect of the current therapy.

### Example 2: shRNA Knockdown Decreases Infection of Human Lung Cells Against Human Coronavirus

MRC-5 (ATCC® CCL-171™) cells were cultured with media, ATCC-formulated Eagle’s Minimum Essential Medium, Catalog No. 30-2003. With fetal bovine serum to a final concentration of 10% at 37° C. in a 5% CO2 incubator. The MRC-5 cell line was derived from normal lung tissue of a 14-week-old male fetus by J.P. Jacobs in September of 1966. The infected MRC-5 cells were infected with Human coronavirus 229E (HCoV 229E VR-740, ATCC® VR-740™) - FIG. 3 control.

In FIG. 3, the HEK293 cells producing shRNA targeting HCoV-229E ORF4a were put into the culture-basket and co-cultured with MRC-5 cell infected with HCoV 229E VR-740 (Co-Culture). HCoV-229E ORF4a is reported to regulate viral production (Ronghua Zhang, Kai Wang, Wei Lv, Wenjing Yu, Shiqi Xie, Ke Xu, Wolfgang Schwarz, Sidong Xiong, Bing Sun, The ORF4a protein of human coronavirus 229E functions as a viroporin that regulates viral production, Biochimica et Biophysica Acta (BBA) -Biomembranes, Volume 1838, Issue 4, 2014, Pages 1088-1095, ISSN 0005-2736, https://doi.Org/10.1016/j.bbamem.2013.07.025.).

[image]

PCR was conducted to amplify the spike protein of HCoV 229E for the detection of the virus in MRC-5 cells utilizing the following primers. [image] As shown in FIG. 3, after co-culture of the MRC-5 cells infected with HCoV 229E VR-740 and HEX293 cells producing shRNA targeting HCoV 229E viral production, viral production was significantly reduced compared to the control.
Lane 1: Ladder
Lane 2: No-sample
Lane 3: HCoV 229E infected MRC-5 Fibroblast
Lane 4: No-sampl
Lane 5-7: HCoV 229E infected MRC-5 Fibroblast co-cultured with HEK293 cells producing shRNA targeting HCoV 229E genome
Lane 8: Ladder

Exosomes were loaded with shRNA using Exo-Fect™ Exosome Transfection Kit (https://systembio.com/shop/exo-fect-exosome-transfection-kit). As shown in FIG. 4, after exposing the MRC-5 cells to exosomes containing shRNA targeting HCoV 229E ORF4a, level of virus was significantly reduced compared to the control, treated with exosomes without shRNA or treated with shRNA alone.
Lane 1: Ladder
Lane 2: HCoV 229E infected MRC-5 Fibroblast (treated with shRNA alone, without exosomes)
Lane 3: HCoV 229E infected MRC-5 Fibroblast (treated with exosomes without shRNA)
Lane 4: HCoV 229E infected MRC-5 Fibroblast (treated with exosomes with shRNA)

The data provided in FIG. 4 clearly shows advantage of implementing intracellular delivery by exosomes in the efficacy of antiviral knockdown agents.

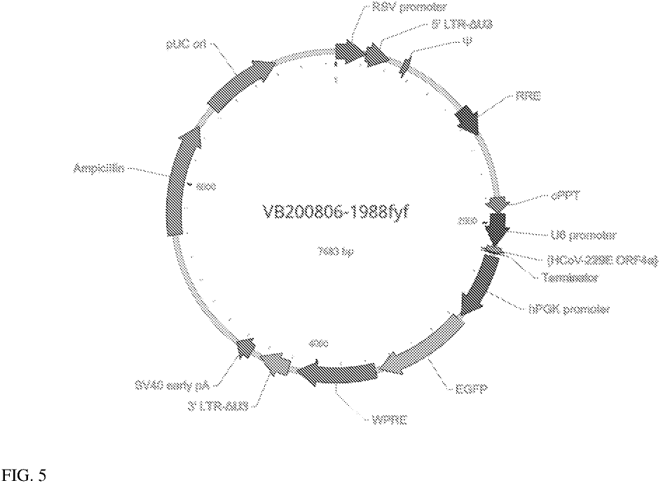

The Vector used in generating the shRNA is shown in FIG. 5. Provided below is Table 1 describing the components of the Vector.

TABLE 1

| Name | Position | Size (bp) | Type | Description | Application notes |
|---|---|---|---|---|---|
| RSV promoter | [symbol] 1-229 | 229 | Promoter | Rous sarcoma virus enhancer/promoter | Strong promoter; drives transcription of viral RNA in packaging cells. |
| 5′ LTR-ΔU3 | [symbol] 230-410 | 181 | LTR | Truncated HIV-15′ long terminal repeat | Allows transcription of viral RNA and its packaging into virus. |
| ψ | [symbol] 521-565 | 45 | Miscellaneous | HIV-1 packaging signal | Allows packaging of viral RNA into virus. |
| RRE | [symbol] 1075-1308 | 234 | Miscellaneous | HIV-1 Rev response element | Rev protein binding site that allows Rev-dependent nuclear export of viral RNA during viral packaging. |
| cPPT | [symbol] 1803-1920 | 118 | Miscellaneous | Central polypurine tract | Facilitates the nuclear import of HIV-1 cDNA through a central DNA flap. |
| U6 promoter | [symbol] 1927-2175 | 248 | Promoter | Human U6 small nuclear 1 promoter | Pol III promoter: drives expression of small RNAs. |
| (MCoV-229E ORF4a) | [symbol] 2178-2221 | 44 | shRNA | Sequence to inhibit ORF4a expression of the Human Coronavirus-2295 | None |
| Terminator | [symbol] 2222-2226 | 5 | terminator | Pol III transcription terminator | Allows transcription termination of small RNA transcribed by Pol III RNA polymerase. |
| hPGK promoter | [symbol] 2254-2758 | 505 | Promoter | Human phosphoglycerate kinase 1 promoter | Medium-strength promoter. |
| EGFP | [symbol] 2789-3508 | 720 | ORF | Enhanced green fluorescent protein: codon optimized based on a variant of wild hype GFP from the jellyfish Aequorea victoria | Commonly used green fluorescent protein: ranked high in brightness, photostability and pH stability among all fluorescent proteins. |
| WPRE | [symbol] 3540-4137 | 598 | Miscellaneous | Woodchuck hepatitis virus posttranscriptional regulatory element | Enhances virus stability in packaging cells, leading to higher titer of packaged virus; enhances higher expression of transgenes. |
| 3′LTR-ΔU3 | [symbol] 4203-4437 | 235 | LTR | Truncated HIV-13′ long terminal repeat | Allows packaging of viral RNA into virus; self-inactivates the S′ LTR by a copying mechanism during viral genome integration: contains polyadenylation signal for transcription termination. |
| SV40 early pA | [symbol] 4510-4544 | 135 | PolyA_signal | Simian virus 40 early polyadenylation signal | Allows transcription termination and polydenylation of mRNA transcribed by Pol II RNA polymerase. |
| Ampicillin | [symbol] 5598-6458 | 861 | ORF | Ampicillin resistance gene | Allows E. coli to be resistant to ampicillin. |
| pUC ori | [symbol] 6529-7217 | 589 | Rep_origin | pUC origin of replication | Facilitates plasmid replication in E. coli: regulates high-copy plasmid number (500-700). |

Note: Components added by user are listed in bold red text.

Provided below is the sequence of the Vector

[symbol]

In reviewing the detailed disclosure which follows, and the specification more generally, it should be borne in mind that all patents, patent applications, patent publications, technical publications, scientific publications, and other references referenced herein are hereby incorporated by reference in this application in order to more fully describe the state of the art to which the present disclosure pertains.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

It is important to an understanding of the present disclosure to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the disclosure as disclosed and claimed herein, the following definitions are provided.

While a number of embodiments of the present disclosure have been shown and described herein in the present context, such embodiments are provided by way of example only, and not of limitation. Numerous variations, changes and substitutions will occur to those of skilled in the art without materially departing from the disclosure herein. For example, the present disclosure need not be limited to best mode disclosed herein, since other applications can equally benefit from the teachings of the present disclosure. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this disclosure as defined in the following claims, in accordance with relevant law as to their interpretation.

What is claimed is:

1. A method for treating or preventing the recurrence of cancer in a subject characterized by having cancer stem cells, said method comprising administering a therapeutically effective amount of a stemness modulating agent to said subject.

2. The method of claim 1, wherein said stemness modulating agent downregulates expression of nanog or Oct4.

3. The method of claim 1, further comprising administering an additional cancer therapy to said subject prior to, during or subsequent to said administering of said stemness modulating agent.

4. The method of claim 3, wherein said additional cancer therapy comprises administering a therapeutically effective amount of a chemotherapeutic agent.

5. The method of claim 2, wherein ceasing expression of nanog or Oct4 causes said cancer stem cell to become a more rapidly dividing cell.

6. The method of any of claims 1-6, wherein the cancer is breast cancer, testicular cancer, lung cancer, melanoma, brain cancer, myeloma, Hodgkin’s disease, hepatoma, stomach cancer, bladder cancer, uterine cancer, neuroblastoma, thyroid cancer, sarcoma, cervical cancer, Wilm’s tumor, colorectal cancer, pancreatic cancer, skin cancer, prostate cancer, ovarian cancer, kidney cancer, lymphoma, acute myelogenous leukemia, acute lymphocytic leukemia, multiple myeloma, ependymoma, chronic lymphocytic leukemia, myelodysplastic syndrome, or chronic myelogenous leukemia.

7. A method of screening for therapeutic agents useful in the treatment of cancer in a mammal comprising the steps of i) contacting a test compound with a cancer stem cell expressing a nanog and/or Oct4 polypeptide and ii) detecting a deleterious effect on said cancer stem cell, wherein a test compound which shows a deleterious effect is identified as a potential therapeutic agent for killing, differentiating or weakening a nanog expressing cancer stem cell.

8. The method of claim 7, wherein said therapeutic agent causes said nanog expressing cancer stem cell to cease expressing nanog.

9. A pharmaceutical composition for the treatment of cancer in a mammal comprising a stemness modulating agent and a pharmaceutically acceptable carrier.

10. Method for the preparation of a pharmaceutical composition useful for the treatment of cancer in a mammal comprising the steps of i) identifying a therapeutic agent in accord with the method of claim 7; ii) determining whether said therapeutic ameliorates the cancer in a mammal; and iii) combining of said therapeutic agent with an acceptable pharmaceutical carrier.

11. A method for preventing, treating, or managing cancer resulting in a reduction in bulk tumor size and/or a reduction in cancer cells, the method comprising identifying the presence of cancer stem cells expressing nanog in a tumor in a human subject, administering to said human subject in need thereof a prophylactically or therapeutically effective regimen, the regimen comprising the administration of a stemness modulating agent to the human subject, and monitoring changes in the amount of said cancer stem cells, wherein the regimen results in at least an approximately 10% reduction in cancer stem cells in said human subject.

12. A method according to any of claims 1-6, wherein the sternness modulating agent is loaded into an exosome.

13. The composition of claim 10, wherein the stemness modulating agent is loaded into an exosome.

14. The composition of claims 10 or 13, further comprising a chemotherapeutic agent.

15. A method for treating a disease or disorder associated with a coronavirus infection in a subject, the method comprising: administering to said subject a therapeutically effective amount of an antiviral knockdown agent.

16. The method of claim 15, wherein the antiviral knockdown agent comprises oligonucleotide-based inhibitor.

17. The method of claim 16, wherein the oligonucleotide-based inhibitor is an RNA antisense molecule, DNA antisense molecule, siRNA, shRNA, dsRNA, miRNA, ribozyme that targets a viral gene from a coronavirus.

18. The method of claim 17, wherein the viral gene encodes for a coronavirus spike protein or comprises coronavirus ORF4.

19. The method of claim 15, wherein the antiviral knockdown agent comprises a gene-editing system.

20. The method of claim 19, wherein the gene-editing system comprises a CRISPR-Cas system.

21. The method of claim 15, wherein the antiviral knockdown agent comprises an antibody or aptamer targeting a viral gene product.

22. The method of any of claims 15-21, wherein the antiviral knockdown agent is formulated in a composition for facilitating intracellular delivery.

23. The method of claim 22, wherein the antiviral knockdown agent is packaged in an exosome or liposome, or is associated with a lipid-based nanoparticle.

24. The method of any of claims 15-23, wherein the coronavirus comprises Sars-CoV-2 or HCoV 229E.

25. The method of any of claims 15-24, further comprising co-administering a therapeutically effective amount of remdesivir, chloroquine, hydroxychloroquine, atazanavir, daclatasvir, sofosbuvir, ganciclovir, foscamet, cidofovir, indinavir, lopinavir, interferon (e.g. interferon-betal), ritonavir, AZT, lamivudine and/or saquinavir.

26. A composition comprising an antiviral knockdown agent packaged in an exosome, liposome, or associated with a lipid-based nanoparticle.

27. The composition of claims 26, wherein the antiviral knockdown agent comprises oligonucleotide-based inhibitor.

28. The composition of claim 27, wherein the oligonucleotide-based inhibitor is an RNA antisense molecule, DNA antisense molecule, siRNA, shRNA, dsRNA, miRNA, ribozyme that targets a viral gene from a coronavirus.

29. The composition of claim 28, wherein the viral gene encodes for a coronavirus spike protein or comprises coronavirus ORF4.

30. The composition of claim 26, wherein the antiviral knockdown agent comprises a gene-editing system.

31. The method of claim 30, wherein the gene-editing system comprises a CRISPR-Cas system.

32. The method of claim 26, wherein the antiviral knockdown agent comprises an antibody or aptamer targeting a viral gene product.

33. The composition of any of claims 26-31, wherein the composition further comprises remdesivir, chloroquine, hydroxychloroquine, atazanavir, daclatasvir, sofosbuvir, ganciclovir, foscamet, cidofovir, indinavir, lopinavir, interferon (e.g. interferon-beta1), ritonavir, AZT, lamivudine and/or saquinavir.
